(19) United States
(12) Patent Application Publication
(10) Pub. No.: US 2026/0102074 A1
(43) Pub. Date: Apr. 16, 2026

(54) THREE-DIMENSIONAL ELECTRICAL IMPEDANCE TOMOGRAPHIC IMAGING

(71) Applicant: CARDIOINSIGHT TECHNOLOGIES INC., MINNEAPOLIS, MN (US)

(72) Inventors: Alfonso SANTIAGO FERRANDEZ, MINNEAPOLIS, MN (US); QING LOU, SOLON, OH (US); Jing ZHAO, SUPERIOR, CO (US); Shuang CHANG, LOUISVILLE, CO (US); Richard L. CROFT, MEAD, CO (US); Cahal MCVEIGH, WHITE BEAR TOWNSHIP, MN (US); Markus W. REITERER, PLYMOUTH, MN (US); William J. PEINE, ASHLAND, MA (US); Qingguo ZENG, SOLON, OH (US); Timothy G. LASKE, SHOREVIEW, MN (US)

(21) Appl. No.: 19/345,577

(22) Filed: Sep. 30, 2025

Related U.S. Application Data
(60) Provisional application No. 63/707,498, filed on Oct. 15, 2024.

(51) Int. Cl. A61B 5/0536; A61B 34/20

(52) U.S. Cl. CPC A61B 5/0536; A61B 34/20; A61B 2034/2051; A61B 2562/046

(57) ABSTRACT

An example system includes a plurality of electrodes and an electrode interface circuit. A controller can control the electrode interface circuit to select which of the plurality of electrodes are source electrodes and which of the plurality of electrodes are measurement electrodes during respective phases of an acquisition cycle. Non-transitory memory can store electrical data representative of measured electrical signals and applied electrical signals for the acquisition cycle. The memory also stores geometry data representative of a three-dimensional position of each of the electrodes relative to the patient's body. A processor can compute an electrical impedance between the source electrode and each of the measurement electrodes based on the electrical data for each of the respective phases of the acquisition cycle. The processor can also construct a three-dimensional tomographic impedance image of the patient's body for the acquisition cycle based on the computed electrical impedances and the geometry data.

### CROSS-REFERENCE TO RELATED APPLICATION

This application claims the benefit of priority to U.S. provisional patent application No. 63/707,498, filed Oct. 15, 2024, which is hereby incorporated herein by reference in its entirety.

### TECHNICAL FIELD

This disclosure relates to three-dimensional electrical impedance tomographic imaging.

### BACKGROUND

Electrical impedance tomography (EIT) is a noninvasive type of medical imaging in which the electrical conductivity, permittivity, and impedance of a part of the body is determined from surface electrode measurements. The impedance and other data can be used to form a tomographic image of that part. EIT is regularly used for healthcare applications such as monitoring lung function in a variety of settings, such as to provide a two-dimensional image for monitoring fluid within lungs.

### SUMMARY

This disclosure relates to three-dimensional electrical impedance tomographic imaging, such as for tumor detection, organ monitoring, electrophysiological mapping. and/or surgical navigation.

As one example, a system includes a plurality of electrodes adapted to be distributed in a three-dimensional arrangement relative to a patient's body. An electrode interface circuit is coupled to each of the plurality of electrodes and configured to one of apply an electrical signal to or measure an electrical signal from each the plurality of electrodes. A controller is configured to control the electrode interface circuit during an acquisition cycle to select which of the plurality of electrodes are source electrodes and which of the plurality of electrodes are measurement electrodes during respective phases of the acquisition cycle, in which each source electrode is configured to apply a source electrical signal to the patient's body and each of the measurement electrodes is configured to measure an electrical signal responsive to the applied electrical signal during a respective phase of the acquisition cycle. Non-transitory memory can store instructions and data, in which the data includes electrical data representative of the measured electrical signals and the applied electrical signals for the acquisition cycle, and geometry data representative of a three-dimensional position of each of the plurality of electrodes relative to the patient's body. A processor coupled to the memory to access the data and instructions stored in the memory, the instructions, when executed by the processor, cause the processor to at least:
compute an electrical impedance between the source electrode and each of the measurement electrodes based on the electrical data for each of the respective phases of the acquisition cycle; and
construct a three-dimensional tomographic impedance image of the patient's body for the acquisition cycle based on the computed electrical impedances and the geometry data.

As another example, a computer-implemented method includes selecting an electrode set that includes at least one source electrode and multiple measurement electrodes from a plurality of electrodes distributed across an individual's body. The method also includes controlling application of an electrical signal to the at least one source electrode of the selected electrode set for a respective phase of an acquisition cycle that includes a plurality phases. The method also includes measuring electrical signals from the measurement electrodes of the selected electrode set responsive to the applied electrical signal for the respective phase of the acquisition cycle. The method also includes repeating the selecting, the controlling and the measuring for each other phase of the acquisition cycle to provide electrical data representative of the applied electrical signals and the measured electrical signals for the acquisition cycle. The method also includes computing, by a processor, an electrical impedance between the at least one source electrode and each of the measurement electrodes based on the electrical data for each phase of the acquisition cycle. The method also includes constructing, by the processor, a three-dimensional tomographic impedance image of the individual's body for the acquisition cycle based on the computed electrical impedances and geometry data, in which the geometry data represents a three-dimensional position of each of the plurality of electrodes distributed across the individual's body.

A non-transitory can store data and instructions that, when executed by a processor, cause the processor to perform a method. The method includes selecting an electrode set that includes at least one source electrode and multiple measurement electrodes from a plurality of electrodes distributed across an individual's body. The method also includes controlling application of an electrical signal to the at least one source electrode of the selected electrode set for a respective phase of an acquisition cycle that includes a plurality phases. The method also includes receiving measured electrical signal from the measurement electrodes of the selected electrode set responsive to the applied electrical signal for the respective phase of the acquisition cycle. The method also includes repeating the selecting, the controlling, and the storing for each other phase of the acquisition cycle to store electrical data representative of the applied electrical signals and the measured electrical signals for the acquisition cycle. The method also includes computing an electrical impedance between the at least one source electrode and each of the measurement electrodes based on the electrical data for each phase of the acquisition cycle. The method also includes constructing a three-dimensional tomographic impedance image of the individual's body for the acquisition cycle based on the computed electrical impedances and geometry data, in which the geometry data represents a three-dimensional position of each of the plurality of electrodes distributed across the individual's body.

### BRIEF DESCRIPTION OF THE DRAWINGS

FIG. 1 depicts an example system to implement three-dimensional electrical impedance tomographic imaging.

FIG. 2 depicts an example of an electrode interface control system.

FIG. 3 depicts an example of a high-resolution three-dimensional image generator.

FIG. 4 depicts an example of a three-dimensional deformation generator.

FIG. 5 depicts an example of a system for combining three-dimensional EIT image and navigation data.

FIG. 6 depicts an example of reconstructing electrophysiological signals based on three-dimensional EIT image data.

FIG. 7 depicts an example of a system that combines surgical navigation and reconstructed electrophysiological signals.

FIG. 8 depicts an example of an arrangement of electrodes configured to be placed on an outer surface of individual's body.

FIG. 9 is a flow diagram of an example method for performing electrical impedance tomographic imaging.

### DETAILED DESCRIPTION

This disclosure relates systems and methods to implement three-dimensional (3D) electrical impedance tomography (EIT) (also referred to as electrical impedance tomographic imaging). As described herein, 3D EIT can be used in a variety of applications, such as for non-ionizing 3D medical imaging, electrophysiological mapping (e.g., electrocardiographic imaging (ECGI)), and/or surgical navigation to name a few.

As an example, an arrangement of electrodes is configured to be distributed across a patient's body, such as surrounding the thorax. An electrode interface circuit is coupled to the electrodes and configured to apply an electrical signal (e.g., current) to one or more electrodes and to measure an electrical signal from other electrodes responsive to the applied electrical signal(s). The electrode interface circuit can operate electrodes as source electrodes or as measurement electrodes in a source mode or a measurement mode, respectively, during a respective phase of a multi-phase EIT acquisition cycle.

A controller is configured to control the electrode interface circuit to select which of the plurality of electrodes are source electrodes and which of the plurality of electrodes are measurement electrodes during respective phases of the acquisition cycle. For example, in a given phase of the EIT acquisition cycle, the controller controls the electrode interface circuit to apply an electrical signal to one or more source electrodes (operating in the source mode) and to measure an electrical signal from each measurement electrode (operating in the measurement mode) responsive to one or more electrical signals applied by one or more respective source electrodes. The controller can also control the electrode interface circuit to sense electrophysiological signals at one or more of the electrodes for measuring native electrophysiological signals or other signals that are not responsive to the electrical signals applied by the source electrodes. The electrode interface circuit can include circuitry (e.g., amplifiers, analog-to digital converter circuitry, switching circuitry) to capture the measured and applied signals and provide electrical data for further processing by a computing apparatus.

The computing apparatus can include non-transitory memory and one or more processors. The memory can store instructions and data, in which the data comprises electrical data representative of the measured electrical signals and the applied electrical signals for the acquisition cycle, and geometry data representative of a three-dimensional position of each of the plurality of electrodes relative to the patient's body. The processor is coupled to the memory and configured to access the data and instructions stored in the memory. The processor thus can execute instructions to compute an electrical impedance (e.g., a complex impedance value having real and imaginary components) between each of the source electrodes and each of the measurement electrodes based on the electrical data for each of the respective phases of the acquisition cycle. The processor can also execute instructions to construct a three-dimensional tomographic impedance image of the patient's body for the acquisition cycle, which can be stored in memory as EIT image data, based on the computed electrical impedances and the geometry data.

In some examples, the 3D EIT image data can be combined (e.g., through registration and other image processing techniques) with high resolution 3D medical image data acquired from another medical imaging modality to provide a high-resolution image or geometry data representative of one or anatomical surfaces (e.g., organ surfaces). Also, or as an alternative, the 3D EIT image data is acquired over time, at a sufficient sample rate (e.g., multiple times per second), so the 3D EIT image data can visualize deformation (e.g., real-time deformation) of anatomical structures, including organ surfaces. For example, the 3D EIT image data can visualize the deformation of the thoracic cavity due to breathing and/or heart deformation.

In a further example, the processor can execute instructions to process (e.g., through image segmentation) the 3D EIT image data and determine a 3D representation of one or more organs or other anatomical structures (e.g., lungs, stomach, liver, intestines and/or heart) and provide a 3D geometry representative of the organ(s). If the organ is the heart, the cardiac geometry (e.g., 3D geometry of endocardial and/or epicardial surfaces) can be used for mapping in electrocardiographic imaging (ECGI), in which the processor executes instructions to reconstruct electrophysiological signal on the cardiac surface based on the cardiac geometry, geometry of the electrodes and electrophysiological signals measured by the electrodes. Thus, the 3D EIT image data enables ECGI to be implemented without ionizing radiation and without requiring pre-surgical medical imaging, which can save both time and cost for patients and providers. The EIT system can also be portable and low cost compared to many existing imaging modalities. The systems and methods described herein further can achieve synergies in some applications (e.g., electrophysiology applications) because the same electrodes implemented for EIT can also (e.g., sequentially or concurrently) acquire electrophysiological signals for monitoring and analyzing the patient's native electrophysiological activity (e.g., for the heart or brain).

In a further example, the 3D-EIT image data can be combined with surgical navigation system, such as electromagnetic (EM) navigation. The processor can execute instructions to process (e.g., through segmentation and registration) the 3D-EIT images and tracking data from the navigation system to visualize, in a common 3D spatial domain, both anatomical structures through which a catheter (or other probe) is traveling and the catheter position relative to such structures. As described herein, the visualization of the anatomical structures can include real-time deformation of such structures based on the acquired 3D-EIT image data. The combination of 3D-EIT imaging and navigation is expected to represent a significant advantage compared with the current two-dimensional cineangiography techniques.

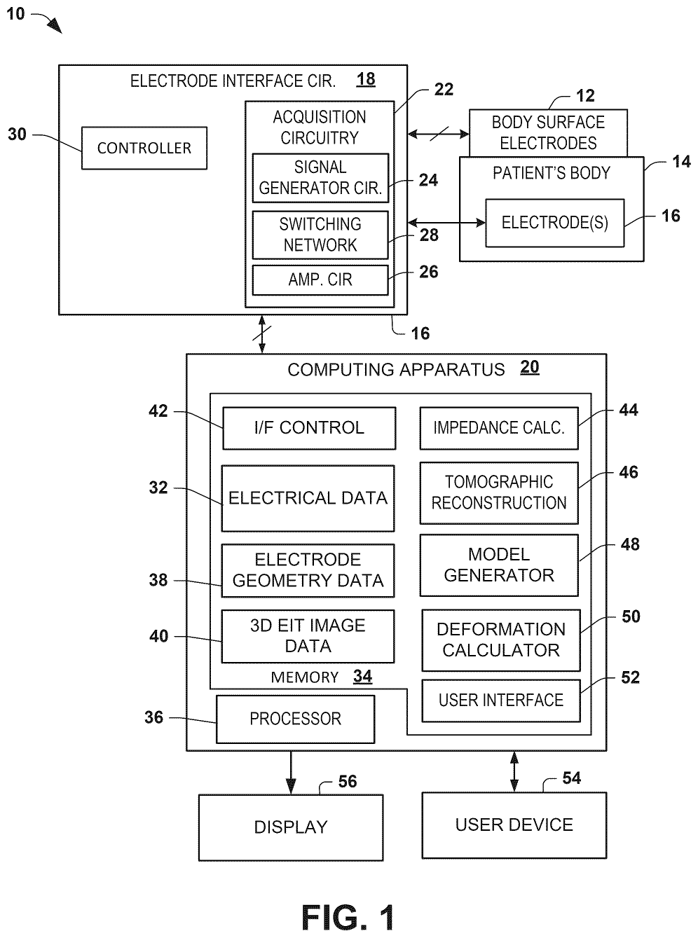

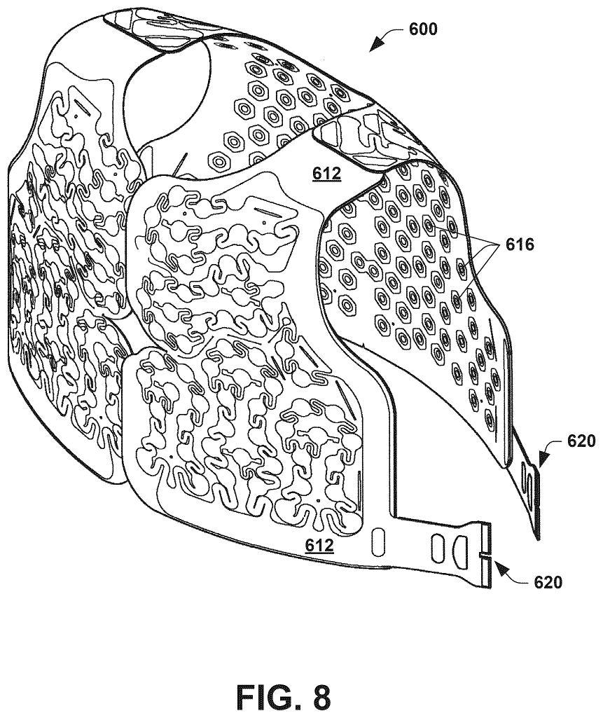

FIG. 1 depicts an example of a system 10 for performing EIT imaging and related functions. The system 10 includes a plurality of electrodes, including an arrangement of body surface electrodes 12 that can be distributed around one or more anatomical regions of interest of a patient's body 14. For example, the body surface electrodes 12 can be carried by one or more conformable substrates (e.g., one or more layers of a web of flexible material) adapted to hold the electrodes at respective sensing locations in contact with the patient's skin (see, e.g., FIG. 8 showing an example vest). In other examples, one or more other forms of conformable substrates (e.g., in the form of patches, strips or a jacket) can be used to attach the electrodes 12 on the patient's body 14, the electrodes 12 can be placed individually on the patient's body, or subsets of the electrodes 12 can be placed in respective groups on the patient's body 14.

In some examples, the system 10 can also include one or more electrodes (also referred to as one or more invasive electrodes, which can be placed as part of the intracavital or endoluminal surgical tool) 16 that can be located within a patient's body 14. The electrode(s) 16 can be carried by a catheter or other type of probe such that the electrode is moveable within the patient's body. Also, or as an alternative, the invasive electrode can be implanted at a location within the patient's body 14, such as part of an implantable medical device. Useful examples of the implantable medical device include an implantable cardioverter-defibrillator, a pacemaker, or a ventricular assist device.

The system also includes an electrode interface circuit 18 and a computing apparatus 20. The electrode interface circuit 18 has input/output (I/O) ports (or terminals) coupled to the electrodes 12 and 16, such as through respective electrically conductive leads. The electrode interface circuit 18 is configured to receive electrical signals from one or more of the electrodes 12 and/or 16 and provide corresponding electrical measurement signals representative of the electrical signals measured by the respective electrodes 12 and/or 16. The acquisition circuitry 22 is also configured to apply electrical signals to one or more of the respective electrodes 12 and/or 16. For example, the applied electrical signals can be an electrical current signal pulse provided as subthreshold pulse (e.g., does not induce an action potential). The electrode interface circuit 18 can also be configured to provide information describing each of the applied electrical signals, such as electrical parameters (e.g., amplitude, frequency, pulse duration, etc.) and timing information.

As a further example, the acquisition circuitry 22 includes signal generator circuitry 24, amplifier circuitry 26, and a switching network 28. The signal generator circuitry 24 can include circuitry configured to apply fields (e.g., inject electrical energy in the form of current or voltage) between the source electrode and each of the measurement electrodes. In an example, the signal generator circuitry 24 can include an instance of a current source (e.g., a controllable current source) for each of the electrodes 12 and/or 16. Alternatively, an instance of a current source can be configured to apply current to multiple source electrodes, consecutively or concurrently. The amplifier circuitry 26 can include an instance of an amplifier for each of the electrodes 12 and/or 16. The switching network (e.g., one or more multiplexers) can have output terminals coupled to the I/Os of the electrode interface circuit 18 for each of the electrodes 12 and 16 and input terminals coupled to signal generator and amplifier for each of the respective electrodes 12 and 16. A controller (e.g. a microcontroller, processor or field programmable gate array (FPGA)) 30 can be configured to control the switching network 28 to selectively couple one or more current sources of the signal generator circuitry 24 with one or more respective source electrodes 12, 16 and selectively couple one or more amplifiers of the amplifier circuitry 26 with one or more other respective measurement electrodes 12, 16. As described herein, each of the electrodes 12 and 16 can operate a source electrode when used to apply an electrical signal or as a measurement electrode when used to receive (e.g., measure) an electrical signal. The measurement electrodes selected (e.g., by the controller 30) for a respective phase of the acquisition cycle can include any number of the electrodes 12 and 16 excluding each source electrode for the respective phase.

For example, the controller 30 selects which of the plurality of electrodes (if any) are source electrodes and which of the plurality of electrodes are measurement electrodes during each phase of a multi-phase acquisition cycle. The number of phases in a given acquisition cycle can depend on the total number of electrodes and the number of source electrodes selected for each respective phase. As described herein, an EIT mode refers to when the system 10, including the electrode interface circuit 18, applies and measures electrical signals for purposes of performing EIT. In some examples, the system 10, including the electrode interface circuit 18, can operate in a measurement mode, in which the controller 30 controls the switching network to only measure electrophysiological signals from the patient's body with all or a selected subset of the electrodes 12 and 16 (e.g., no electrical signals are applied). The system 10, including the electrode interface circuit 18, can implement the EIT and measurement modes consecutively or concurrently.

In one example, the controller 30 controls the switching network 28 for a given phase of the acquisition cycle to apply electrical current signals from the signal generator circuitry 24 to a given one of the electrodes 12 and 16 and to measure electrical signals (e.g., voltage signals) from each of the measurement electrodes for the given phase. The controller 30 can control the switching network 28 (and other circuitry) to apply an electrical current signal to each other respective source electrode 12, 16 sequentially in subsequent respective phases of the acquisition cycle measuring electrical signal from respective measurement electrodes 12, 16 concurrently responsive to the applied electrical current signal applied during each respective phase.

In another example, the controller 30 controls the switching network 28 for a given phase of the acquisition cycle to apply electrical current signals from the signal generator circuitry 24 having different frequencies concurrently to multiple source electrodes (e.g., a selected subset of two or more source electrodes) 12 and 16 and to measure electrical signals (e.g., voltage signals) from each of the measurement electrodes for the given phase. By injecting current with multiple frequencies from different source electrodes concurrently, the time for a given acquisition cycle can be reduced. The controller 30 can control the switching network 28 (and other circuitry) to apply electrical current signals having different frequencies concurrently to other subsets of two or more source electrodes during each subsequent phases of the acquisition cycle while also measuring electrical signal from respective measurement electrodes concurrently responsive to the applied electrical current signal during each phase. The number of source electrodes can be the same in each phase of the acquisition cycle or the number of source electrodes can vary from phase to phase.

In the example of FIG. 1, the electrode interface circuit 18 is coupled to the computing apparatus 20 through a communication link, which can be a physical link or a wireless link. The electrode interface circuit 18 can provide electrical data 32 to the computing apparatus 20 through the link, which can be stored in memory 34 of the computing apparatus. While the example of FIG. 1 shows the electrode interface circuit 18 and a computing apparatus 20 as separate structures, in other examples, the electrode interface circuit 18 and computing apparatus 20 can be implemented in a single structure (e.g., containing both hardware and software components). The electrode interface circuit 18 can include an analog-to-digital converter (not shown) to convert analog signals to respective digital signals. The electrical data 32 thus can include digital information representing each applied electrical signal (e.g., current) and each electrical signal measurement (e.g., measured voltage) for each phase of each acquisition cycle. The electrical data 32 further can include channel information (e.g., for each electrode), timestamps, and other parameters associated with each applied or measured signal that is acquired.

The memory 34 can be implemented as one or more non-transitory machine readable media, which may be resident in the computing apparatus 20 or remotely located from the computing apparatus (e.g., cloud memory). The memory can include data and instructions executable by a processor 36. The processor 36 can be coupled to the memory 34 to access the data and instructions (e.g., program code) stored in the memory. The instructions, when executed by the processor 36, cause the processor to perform respective control logic, functions, and/or methods described herein.

The processor 36 may include any one or more of a microprocessor, a controller, a digital signal processor (DSP), an application specific integrated circuit (ASIC), a field-programmable gate array (FPGA), or equivalent discrete or integrated logic circuitry. In some examples, processor 36 may include multiple components, such as any combination of one or more microprocessors, one or more controllers, one or more DSPs, one or more ASICs, or one or more FPGAs, as well as other discrete or integrated logic circuitry. The term “processor” may generally refer to any of the foregoing logic circuitry, alone or in combination with other logic circuitry, or any other equivalent circuitry.

In the example of FIG. 1, the data includes the electrical data 32, electrode geometry data 38, and 3D EIT image data 40. Also, in the example of FIG. 1, the executable instructions in the memory 34 include an interface control 42, an impedance calculator 44, a tomographic reconstruction method 46, a model generator 48, a deformation calculator 50, and a user interface 52. Other data as well as other functions and methods can also be stored in the memory 34.

The processor 36 can execute the interface control 42 to provide instructions to the controller 30 of the electrode interface circuit 18, such as to define an operating mode (e.g., EIT or measurement mode) for the system 10 and associated operating parameters. For example, the interface control 42 can provide control instructions to specify which electrodes 12 and 16 are source electrodes and measurement electrodes for each phase of an acquisition cycle as well as parameters (e.g., amplitude, frequency, pulse duration, etc.) of each applied electrical current signal. These parameters will be selected having into account other therapies used during the procedure to avoid interference. The area in which the image will be captured can be also selected through the interface control 42 and parameters can be adjusted accordingly, such as to provide a higher resolution and/or higher refresh rate for that specific field of view (FOV). The interface control 42 can also provide control instructions to set a sample rate for signal measurements (e.g., by measurement electrodes) and gain values for respective amplifiers in the amplifier circuit 26. The interface control 42 further can be programmed to allow a user to place virtual electrodes in the patient thorax (or at other specified locations to also capture classical biopotentials) such as representative of a 12-lead EKG. The sample rate for signal measurements can depend on the acquisition cycle rate, which defines the duration (or rate) for each phase of a given acquisition cycle.

The acquisition cycle rate can be fixed or be variable, such as can be modified during the EIT mode (e.g., by the interface control 42). In an example, the interface control 42 can modify the acquisition cycle rate based on one or more sensed conditions, such as responsive to detecting a change in impedance values in a region of the patient's body and/or a location of the one or more invasive electrodes 16. Also, or as an alternative, the interface control 42 can modify the acquisition cycle rate responsive to a user input instruction provided at a user device 54 to the user interface 52 (e.g., a graphical user interface). For example, each acquisition cycle, which includes a plurality of acquisition phases, can occur at a rate that is less than one cycle per second (e.g., milliseconds) to enable real-time (or near real-time) EIT, as described herein.

The processor 36 can execute the impedance calculator 44 to calculate electrical impedance values between the source electrode and each of the measurement electrodes based on the electrical data 32 for each of the respective phases of an acquisition cycle. The impedance values can represent the electrical impedance (e.g., a measure of the electrical impedance spectrum between each electrode pair) throughout the conductive volume defined by the region of the body 14 surrounded by the body surface electrodes 12. The impedance calculator 44 can compute impedance values continually based on electrical data 32 acquired over time (e.g., over a number of acquisition cycles) for each of the source and measurement electrodes, such as to provide real-time (or near real-time) electrical impedance values. Alternatively, impedance calculator 44 can compute impedance values as static values over one acquisition cycle. A location or region for each computed impedance value can also be provided based on the locations of respective electrodes, such as defined by the electrode geometry data 38.

As an example, the electrical data 32 can represent the applied electrical current signal at one or more source electrodes and the electrical signal measurements at each of the measurement electrodes. The applied electrical current can be stored in the electrical data 32 as a complex current value having real and imaginary components (e.g., magnitude and phase) and the electrical signals (e.g., voltage) measured at the respective measurement electrodes likewise can be stored in the electrical data 32 as a complex voltage value having real and imaginary components (e.g., magnitude and phase). Thus, the impedance calculator 44 can compute a value of electrical impedance as a complex impedance value having real and imaginary components (e.g., magnitude and phase) as a function of complex current and complex voltage values (e.g., Z=V/I). Also, or as an alternative, the impedance calculator 44 can compute the impedance values having only real components based on the applied electrical signal(s) and the electrical signals measured responsive to the applied electrical signal(s).

Each source electrode and measurement electrode can define an electrode pair having an impedance between the respective electrodes (e.g., electrodes distributed across the patient patient's body). The impedance calculator 44 thus can compute an impedance value along a virtual line in 3D space for the respective signals in the electrical data 32 for each source-measurement electrode pair at each phase of each acquisition cycle. The acquisition cycle rate can be fixed or variable, such as described herein.

As a further example, a plurality of the body surface electrodes 12 (e.g., 100 electrodes, 200 electrodes, 250 electrodes or more) are evenly distributed across and surrounding the thorax of the patient's body 14. The density and position of the electrodes on the body surface can depend on the application and, in some examples, there can be predetermined electrode-carrying substrates (e.g., strips, patches, vests or jackets) configured to provide electrodes at desired locations and with a density for respective applications. The impedance calculator 44 can determine a multitude of impedance values across the transthoracic cavity to provide impedance data for each acquisition cycle representative of three-dimensional impedance characteristics for the transthoracic volume of the patient's body 14. In some examples, the interface control 42 can command the electrode interface circuit 18 to vary parameters (e.g., magnitude and/or frequency) of the signals applied by one or more source electrode 12, 16. For example, variations in the amplitude and frequency of electrical signals applied by the source electrodes, which are measured by the respective measurement electrodes, can provide information relative to variations in transthoracic impedance, such as to facilitate identification of different types of tissue and/or tissue boundaries (e.g., organ surfaces).

The processor 36 can execute the tomographic reconstruction method 46 to construct a 3D tomographic impedance image of the patient's body for the acquisition cycle based on the computed electrical impedance values (e.g., computed by the impedance calculator 44) and the electrode geometry data 38. As described herein, the electrode geometry data 38 can represent a spatial position (e.g., 3D spatial coordinates) of the electrodes 12 and 16. The spatial position for each of the electrodes 12 and 16 (as provided by the electrode geometry data 38) can be linked to and/or stored with the electrical data 32 in the memory 34.

The electrode geometry data 38 can be ascertained in a number of ways. In one example, the location and geometry of body surface electrodes 12 are determined by placing the electrodes on a patient during CT scan, MRI scan, SPECT, PET, or another 3D medical imaging modality. Also, or as an alternative, the 3D spatial position of the body surface electrodes 12 can be derived from optical images, such as obtained from a range imaging camera and/or monoscopic imaging device. Also, or as an alternative, the 3D spatial position of the body surface electrodes 12 can be estimated from measurements or data acquired from a from a digitizer that can provide a 3D spatial location for a respective electrode when brought into contact with the respective electrode. Other approaches can also be used to provide the geometry data for the body surface electrodes (e.g., manual measurements, electromagnetic navigation, etc.).

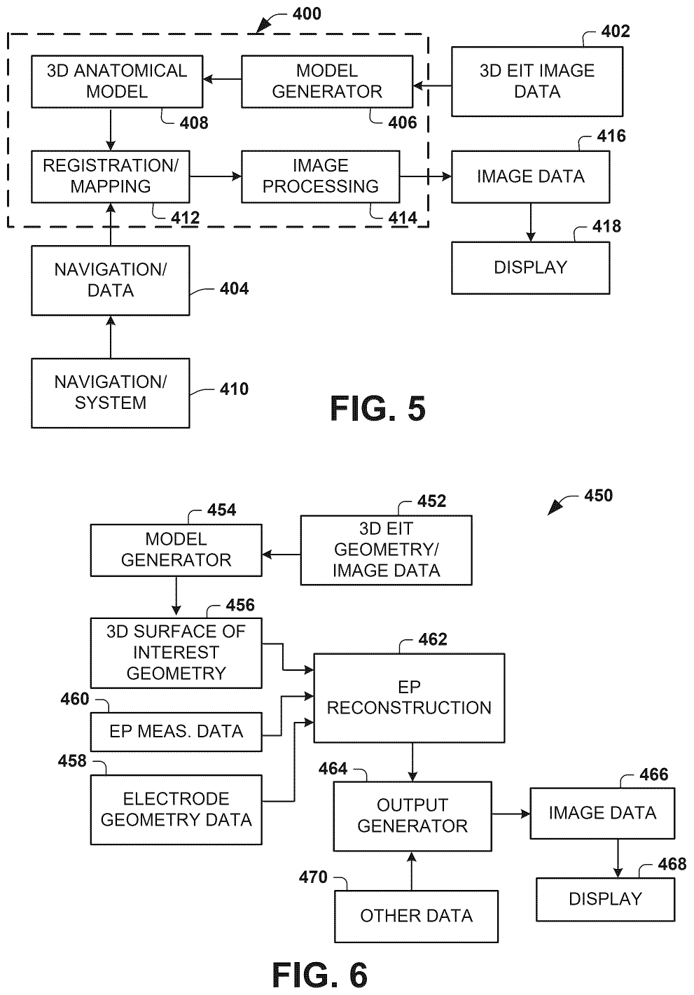

In examples where one or more invasive electrodes 16 are used as source electrodes and/or measurement electrodes, the electrode geometry data 38 also includes data specifying the 3D spatial location of the one or more invasive electrodes 16. For example, the data specifying the 3D spatial location for the invasive electrode 16 can be provided by a spatial tracking system, such as an electromagnetic tracking system, a magnetic tracking, an optical tracking system, or other navigation system, such as described herein (see, e.g., FIG. 5). In some examples the same tracking system can be used to provide the electrode geometry data 38 for both the body surface electrodes 12 and the invasive electrode(s) 16. The electrode geometry data 38 representing the 3D spatial location of the one or more invasive electrodes 16 can be updated at a refresh rate and/or in response to detecting changes in position invasive electrode(s). The electrode geometry data 38 representing the 3D spatial location for the body surface electrodes 12 and the invasive electrodes 16 can be registered in a common spatial coordinate system by applying a corresponding spatial transformation.

The tomographic reconstruction method 46 can construct the 3D tomographic impedance image as an absolute (e.g., static) EIT image or a time-difference EIT image, which is representative of a difference between tomographic impedances at multiple times and respective (e.g., different) physiological states. Alternatively, the tomographic reconstruction method 46 can construct the 3D tomographic impedance image dynamically, in which the 3D tomographic impedance image is updated (e.g., repeatedly constructed in whole or in part) at a refresh rate based on impedance values computed over time. The 3D tomographic impedance image(s) of the patient's body 14 can be stored in the memory 34 as 3D EIT image data 40.

As an example, the tomographic reconstruction method 46 can be programmed to implement one or more tomographic reconstruction methods. The tomographic reconstruction methods can be classified depending on the hypothesis used for the reconstruction and encompassing techniques, such as: back-projection algorithms, linearized inverse algorithms, sensitivity matrix-based methods, gauss-newton methods, conjugate gradient methods, total variation regularization, level set methods, optimization based methods, convolutional neural networks, model-based machine learning, or Bayesian reconstruction. Independently of the reconstruction method implemented by the tomographic reconstruction method 46, the final result is a 3D map (e.g., a heat map) representative of the patient conductivity throughout the volume of the patient's body that is surrounded by the electrodes 12. The 3D map can be stored in the memory 34 as the 3D EIT image data 40. The tomographic reconstruction method 46 may use the entire impedance spectrum, a single frequency band, multiple frequency bands, or a single frequency, and the frequency or frequency band can be defined by the user (e.g., in response to a user input through the user interface 52). When the entire impedance spectrum is not used, the tomographic reconstruction method 46 can be configured to generate a number of different heat maps depending on the frequency band that is being used, each of which can be store as part of the 3D EIT image data 40, analogue to the different weighting images during an MRI. The tomographic reconstruction method 46 further can leverage numerical methods, machine learning, or a combination thereof to as part of image processing to enhance the reconstruction of for providing the 3D EIT image data 40.

As a further example, because the electrodes 12 and 16 can be a 3D arrangement of electrodes around the patient's body (e.g., non-coplanar sets of electrodes surrounding the thorax), the tomographic reconstruction method 46 can be programmed to create the tomographic images based on electrical data for the non-coplanar arrangement of electrodes. Thus, in contrast to reconstructing only along a transversal plane, as is the case with many existing EIT systems, the tomographic reconstruction method 46 can be programmed to reconstruct geometry within the patient's body at any planes (coronal or sagittal and random angle) or as 3D image. In some examples, the tomographic reconstruction can reconstruct geometry within the patient's body as one of a 3D image or along a user-selected plane responsive to a user input instruction provided at the user device 54 to the user interface 52.

The processor 36 can execute instructions to render an image on a display 56 based on the 3D EIT image data 40. The form and content of the image can depend on additional processing of the 3D EIT image data that is implemented and can further be controlled responsive to user input instructions provided at the user device 54 to the user interface 52. In an example, the 3D EIT image data can visualize internal structures of the thorax, abdomen, or both the thorax and abdomen according to the region where the electrodes are placed around.

As one example, the processor 36 can execute the model generator 48 to segment the 3D EIT image data 40, which is produced by the tomographic reconstruction method 46 to provide anatomical data representative of 3D geometry for one or more surfaces of interest within the patient's body. The anatomical data can be provided (e.g., by the model generator 48) as a 3D model that represents the geometry for one or more anatomic surfaces of interest for a given time instance (e.g., a static representation of 3D anatomical geometry based on electrical data for a single acquisition cycle). Also, or as an alternative, anatomical data can represent the anatomical geometry as multiple 3D models for one or more anatomic surfaces of interest multiple acquisition cycles, such that the geometry for each one or more such surfaces include respective models for different acquisition cycles throughout one or more time intervals. As yet another alternative, a generic model of anatomical geometry can be stored in the memory, and the EIT image(s) can be registered with the generic model or a modified version of the generic model that is customized for the patient based on physical measurements and/or EIT image data 40 generated for the patient. The anatomic surfaces of interest can include surfaces of internal organs, such as heart, lungs, and/or vessels, as well as other anatomy, such as, esophagus, trachea, bones, etc. The surface of the skin can be defined based on the location of the body surface of electrodes 12. The model generator 48 can represent the geometry of the respective surface(s) of the model as a 3D mesh including a plurality of nodes having spatial coordinates interconnected by edges to define the mesh. For example, the mesh can be implemented as a triangular (or other polygon) mesh that interconnects the nodes across each surface of interest.

As a further example, the processor 36 can execute the deformation calculator 50 to repeatedly construct the three-dimensional tomographic impedance image based on electrical data 32 acquired (e.g. continuously at a sample rate) over each of a plurality of acquisition cycles. The dynamic 3D EIT image thus can vary over time to represent movement and/or deformation of structures within the patient's body over at least one time interval.

In some examples, the interface control 42 can control a refresh rate at which the 3D EIT image is constructed. The refresh rate can be set based on a user input instruction provided at the user device to the user interface 52. Also, or as an alternative, the interface control 42 can set the refresh rate based on detected movement and/or deformation determined (e.g., by the deformation calculator 50) for one or more structures within the patient's body. Also, or as an alternative, the interface control 42 can adjust the refresh rate based on a location of an object (e.g., the invasive electrode and/or a device carrying the invasive electrode) within the patient's body, in which the location of the object within the patient's body is determined based on analyzing of the 3D EIT image (e.g., image processing, such as segmentation and/or feature extraction). The acquisition rate thus can be set (e.g., by the interface control 42) to enable the deformation calculator 50 to provide EIT images in substantially real time (e.g., at approximately ten image frames per second). The time period for each acquisition cycle can be set to be less than one second (e.g., in the range of tens or hundreds of milliseconds) to provide a 3D image showing movement and/or deformation of the anatomical structures.

In some examples, the invasive electrode 16 and/or a device carrying the invasive electrode can be visualized in the EIT image, such as to track movement of the invasive electrode 16 and/or the device carrying the invasive electrode relative to surround anatomy. As a further example, the processor 36 can execute the model generator 48 to generate a model representative of the invasive electrode 16 and/or the device carrying the invasive electrode and render a graphical representation thereof on the display relative to one or more surfaces of interest (e.g., anatomical surface models) thar are also produced by the model generator 48.

In some circumstances, the usefulness of the 3D EIT image represented by the 3D EIT image data 40 (e.g., as produced by the tomographic reconstruction 46) can depend on the level of contrast and resolution of the image(s). For example, a high resolution 3D EIT image can provide greater utility than a low resolution image, which is at a resolution below a resolution threshold sufficient for visualizing one or more surfaces of interest. As per a general rule, the resolution of the EIT image is proportional to the distance between the electrodes. Therefore, to obtain a higher resolution in the raw EIT image, a higher electrode density can be used. For the example of organ specific applications (e.g., heart, lungs, liver, etc.), the body surface electrodes can include a higher electrode density around the organ of interest. The raw EIT image generated for such an organ-specific application can be used to extract organ deformation from a series of EIT images and provide an organ deformation field (e.g., a deformation vector field). The organ deformation field can be combined with a high resolution medical image technique, such as MRI or x-ray CT, such as described herein.

The processor 36 further can execute instructions (e.g., resolution calculator code) to calculate a resolution of the 3D tomographic impedance image defined by the 3D EIT image data. If the calculated resolution is below a resolution threshold, the image can be enhanced by one or more image enhancement methods executed by the processor 36. In one example, the resolution can be enhanced by combining respective EIT images at different time instances to provide an aggregate image having an increased number of pixels or voxels from a single image. Each of the images being combined can be gated to a common physiological signal (e.g., respiration, cardiac cycle, or the like) to increase the likelihood that the organs are located in the same locations in each of the images being combined. Image segmentation can also be performed to increase the contrast between surface boundaries (e.g., inner and outer surfaces) of respective anatomic structures.

Alternatively, or additionally, the processor 36 can execute instructions to combine the 3D EIT image, which is defined by the 3D EIT image data 40, with other 3D medical imaging data to define a fused 3D image of patient anatomy. The 3D medica medical imaging data can be representative of one or more 3D medical images of a portion of the patient's body acquired by a three-dimensional medical imaging modality, which at includes or at least overlaps with the portion of the patient's body 14 surrounded by the body surface electrodes 12 and represented in the 3D EIT image data 40. Examples of 3D imaging modalities include ultrasound, computed tomography (CT), 3D Rotational angiography (3DRA), magnetic resonance imaging (MRI), x-ray, 3D ultrasound, single-photo emission computed tomography (SPECT), positron emission tomography (PET), fluoroscopy and the like. For example, an image registration algorithm can be executed by the processor 36 applying a spatial transformation to register the 3D medical image and the 3D EIT image in a common 3D spatial coordinate system. Anatomical landmarks and/or other fiducial points can be used to register the 3D medical image and the 3D EIT image. As an example, the landmarks and/or other fiducial points reside on one or more surfaces (e.g., surface of organs) in the 3D model provided by the model generator 48. The landmarks and/or other fiducial points can be identified automatically and/or responsive to user input instructions through the user interface 52 identifying such landmarks and/or other fiducial points in each of the respective images.

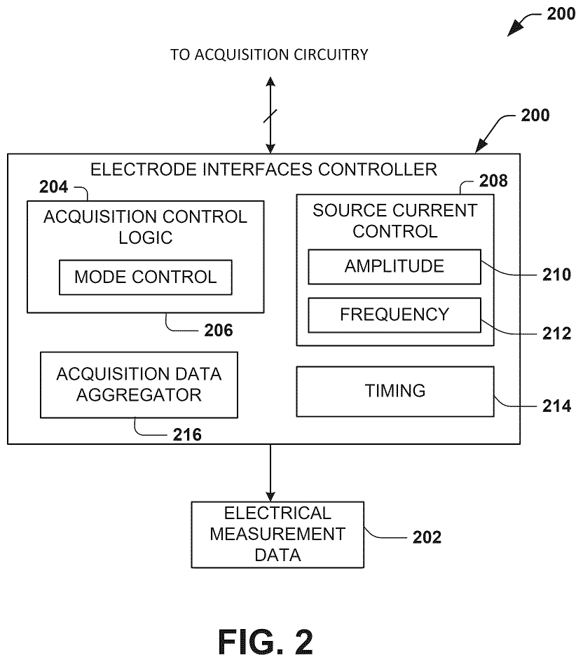

FIG. 2 depicts an example of an electrode interface controller 200. The electrode interface controller 200 is a useful example of the controller 30 shown in FIG. 1, which can control the acquisition circuitry 22 and provide electrical data 202 (e.g., corresponding to the electrical data 32). The electrode interface controller 200 can be implemented as a microcontroller, a control unit, a processor, an FPGA, or the like configured to control the acquisition of electrical signals for use in providing EIT image data described herein. The electrode interface controller 200 can be coupled to acquisition circuitry (e.g., acquisition circuitry 22) and the acquisition circuitry can be coupled to each of the electrodes (e.g., electrodes 12, 16) through an arrangement of switches (e.g., forming switching network 28) and respective connectors for the electrodes.

In the example of FIG. 2, the electrode interface controller 200 includes an acquisition control logic 204 configured to control high-level acquisition functions. For example, the acquisition control logic can include a mode control logic 206 configured to control an operating mode for each of the electrodes (e.g., electrodes 12 and 16). The operating mode can define whether a given electrode 12, 16 is a source electrode or a measurement electrode, which can vary during each phase of a respective acquisition cycle. The mode control logic 206 can be configured to set the operating modes for the electrodes in response to control instructions. For example, a computing apparatus (e.g., interface control 42 of the computing apparatus 20) can provide control instructions to configure the mode control logic 206 and/or the acquisition control logic, more generally, to define the operating modes for injecting and measuring electrical signal for EIT as described herein.

In a first example, the mode control logic 206 sets one electrode as a source electrode and the remaining electrodes (or a subset thereof) as measurement electrodes in a given phase to provide respective measured electrical signals (e.g., voltages) responsive to the signal applied by the source electrode. The process can be repeated during each phase of each acquisition cycle (e.g., sequentially) to inject and collect measurements from different sets of the electrodes.

In a second example, the mode control logic 206 controls multiple electrodes (e.g., at multiple locations across and/or within the patient's body) to operate as source electrodes configured to inject current at different respective frequencies in a given phase of the acquisition cycle. In the second example, the other electrodes (or a subset thereof) can operate as measurement electrodes to provide respective measured electrical signals (e.g., voltages) responsive to the signals applied by each of the source electrodes. Additionally, in the second example, a given electrode can operate as a source electrode, concurrently or sequentially in a given phase, at one frequency and as a measurement electrode for measuring signals at one or more other frequencies. Alternatively, the mode control logic 206 controls a subset of the electrodes to operate as source electrodes only while another subset up to all remaining electrodes operate as measurement electrodes only for respective phase of each acquisition cycle. In either approach, according to the second example, the time for each acquisition cycle and the number of phases can be reduced compared to systems that that use a source electrode injecting a signal at a single frequency.

The electrode interface controller 200 also includes a source current control 208 configured to define operating parameters for each source electrode, which can include the amplitude parameter 210 and/or frequency parameter 212. The mode control logic 206 can define the same parameters for each source electrode during each acquisition cycle. Alternatively, the mode control logic 206 can modify the parameters 210, 212 for each source electrode from one acquisition cycle to the next acquisition cycle. Variations in the frequency of the current injected by source electrodes are expected to enable impedances throughout the 3D body volume to be differentiated more accurately in the EIT images being generated over time.

For example, the mode control 206 of the acquisition control logic 204 is configured to define the amplitude and frequency for current injected by each one or more source electrode during each phase of each acquisition cycle. In an example, the mode control logic 206 can set the amplitude parameter 210 for the injected current to be a low (e.g., subthreshold) current having a positive or negative magnitude that does not create an action potential when injected by the respective source electrode on the body surface (e.g., electrodes 12) or by an invasive electrode within the patient's body (e.g., electrodes 16). The mode control 206 can also set the frequency parameter 212 of the electrical current signal (or signals) that is provided for each phase of the acquisition cycle. The frequency of the current being injected can be in a frequency range outside of line noise (e.g., greater than 60 Hz) and any electrophysiological signals of interest (e.g., outside of 0.5-500 Hz). In an example, the mode control 206 can set the frequency parameter for a given source electrode to inject current with a frequency that is approximately 1 KHz or greater. In examples where multiple source electrodes inject current concurrently during a given phase of the acquisition cycle, the mode control 206 can define the frequency for each injected current signal to provide a bandwidth (e.g., spacing) between each of the injected current signals to reduce interference between signals being injected and measured.

The electrode interface controller 200 also includes a timing circuit 214 configured to control the period (e.g., interval) and duty cycle of respective electrical current signal pulses being injected. The timing circuit 214 can also control the sample rate at which the electrical signals are being measured for each phase of each acquisition cycle, which further can define the duration of each acquisition cycle. In an example, the duration of each phase in a given acquisition cycle is set to enable an acquisition cycle time that is less than one second (e.g., 200 ms, 100 ms, 50 ms, 10 ms or less). Utilizing a larger number of source electrodes to inject current concurrently at different frequencies during each phase can help shorten the acquisition time such that changes in impedance over time can measured with higher granularity over time.

The electrode interface controller 200 also includes an acquisition data aggregator 216 that receives the electrical signals measured by each of the measurement electrodes and each of the one or more current signal(s) injected by each of the source electrodes for each phase. The acquisition data aggregator 216 can provide electrical data 202 (e.g., corresponding to the electrical data 32 of FIG. 1). For example, acquisition data aggregator 216 includes analog-to-digital conversion (ADC) circuitry, such as an ADC for each channel, configured to provide a digital representation of the electrical signals measured by each of the measurement electrodes and the one or more current signal(s) injected by each of the source electrodes. The acquisition data aggregator 216 can also add metadata identifying each channel and timing information (e.g., timestamps, phase and/or acquisition cycle). In some examples, the acquisition data aggregator 216 or other circuits (e.g., in the acquisition circuitry 22) can perform signal processing (e.g., filtering, Fourier transforms, and the like). For example, the amount and type of filtering can vary depending on the location and type of interference noise relative to the electrodes and frequencies being measured by the respective electrodes. Typical filtering involves a band-pass filter to remove any other signal outside of the spectrum of interest, such as 50 Hz or other frequencies that may emitted in close spatial proximity to the patient in the intervention room. Ultimately, the acquisition data aggregator 216 or other circuits perform such filtering to isolate electrical signal components (e.g., voltage signals at respective frequencies) from measured electrical signals responsive to each of the respective injected current signals. Also, or as an alternative, the measured electrical signals can be filtered by software instructions (e.g., code executed by the processor 36) to extract and isolate the electrical signal components (e.g., voltage signals at different respective frequencies) from measured electrical signals for each respective phase of the acquisition cycle. The signal processing can determine the frequencies for extraction and isolation based on the frequency parameters 212 defined for the source electrodes at each phase of the acquisition cycle.

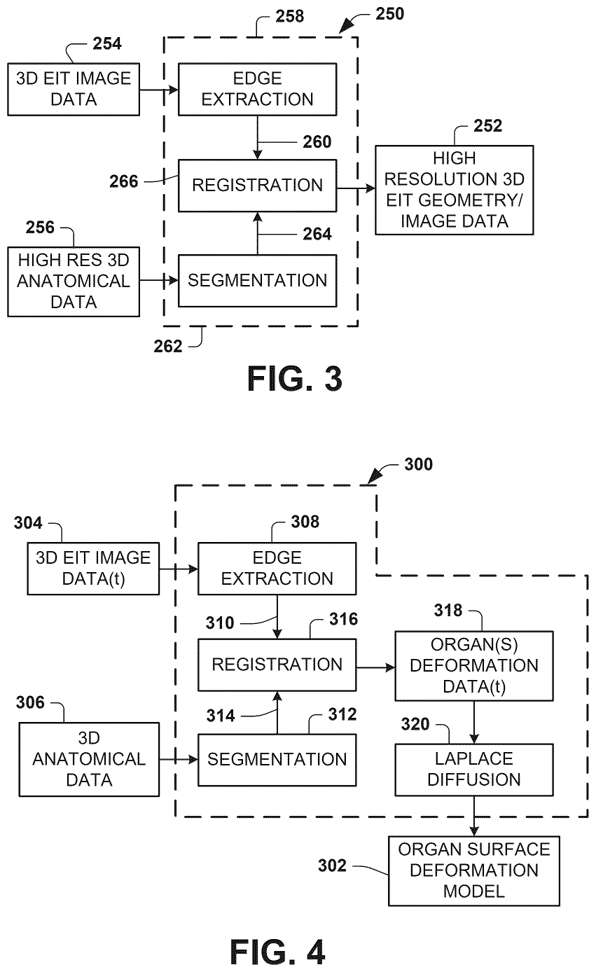

FIG. 3 depicts an example of a high-resolution 3D image generator 250 that can be implemented (e.g., as instructions executable by the processor 36) to provide high-resolution 3D EIT geometry and/or image data (also referred to as geometry/image data) 252. In the example of FIG. 3, the image generator 250 is programmed to provide the high-resolution 3D EIT geometry and/or image data 252 based on 3D EIT image data 254 (e.g., the 3D EIT image data 40) and high-resolution 3D anatomical data 256. The high-resolution 3D anatomical data 256 can include a set of one or more 3D images acquired by a medical imaging modality (e.g., CT, MRI, ultrasound, PET, SPECT, etc.) for a region of interest of the patient's body that resides within the volume represented by the 3D EIT image data 254. Alternatively, high-resolution 3D anatomical data 256 can include a processed image or an anatomical model derived from one or more 3D images acquired by the 3D medical imaging modality. Other types of high-resolution 3D anatomical data 256 can be used in other examples.

The image generator 250 includes an edge extraction method 258 (e.g., instructions executable by the processor 36) to process the 3D EIT image data 254 and to identify edges within the 3D images, such as are representative of boundaries between different tissue structures and/or bodily fluids (e.g., organ surfaces, vessel walls, or the like). For example, the edge extraction method 258 can be programmed to implement a variety of one or more edge detection methods, including search-based and/or zero-crossing based methods (e.g., thresholding, edge thinning, differential approaches, phase stretch transform, or the like) to provide edge-detected image data at 260 (which can be stored in memory for further processing). In some examples, the edge extraction method 258 can include an image segmentation method, such as described herein below, to identify and encode tissue boundaries within the 3D volume defined by the 3D EIT image data. The edge extraction method 258 can also be programmed to perform smoothing of the edge detected data to provide smoothed version of the edge-detected image data at 260.

The image generator 250 also includes an image segmentation method 262 (e.g., instructions executable by the processor 36) to process the high-resolution 3D anatomical data 256 and provide segmented image data 264 (which can be stored in memory for further processing). In some examples, the high-resolution 3D anatomical data 256 can be 3D image for a region of interest of the patient's body, which can be acquired previously and for another purpose. For example, the image segmentation method 262 can implement thresholding method, a clustering method, a histogram-based method or the like on the high-resolution 3D anatomical data 256. Other image segmentation methods can be used in other examples.

The spatial resolution of the 3D EIT image data 254 as well as the edge-detected image data 260 derived therefrom is generally low (e.g., greater than 0.1 μm, such as in the millimeter or centimeter range). Thus, the image generator 250 can be used to increase the spatial resolution of the 3D EIT image data 254 as well as the edge-detected image data 260 derived therefrom. In the example of FIG. 3, the image generator 250 includes a registration method 266 (e.g., instructions executable by the processor 36), such as scale-invariant feature transform, speeded-up robust features, orient FAST and rotated BRIEF feature detector, Harris Corner detector, affine-Invariant feature detector, normalized cross-correlation, sum of squared differences, or other statistical based methods (e.g., deep learning approaches). The registration method 266 is programmed to register the lower resolution edge-detected image data 260 with the higher resolution segmented image data 264 to provide the high-resolution 3D EIT geometry and/or image data 252. The high-resolution 3D EIT geometry and/or image data 252 thus can preserve the tissue boundaries, including organ surfaces and vessel walls, from a current (e.g., more recent or real-time) EIT image data 254, which is enhanced by the higher spatial resolution of the high-resolution 3D anatomical data 256. In some examples the registration method 266 can utilize neural networks (e.g., trained convolutional neural network (CNN)) or other machine learning techniques to facilitate alignment and infer details between the image data sets 260 and 264 (e.g., obtained using different imaging modalities) for generating the high-resolution 3D EIT geometry and/or image data 252. The resulting high-resolution 3D EIT geometry and/or image data 252 can be rendered (e.g., by the processor 36) on an output device (e.g., display 56) for analysis by health care provider. Also, or as an alternative, the high-resolution 3D EIT geometry and/or image data 252 can be stored in memory for analysis by an automated methods of image analysis and/or for further processing, such as described herein. For example, the high-resolution 3D EIT geometry and/or image data 252 can be used to generate a 3D anatomical model for surgical navigation (see, e.g., FIG. 5) and/or for reconstruction of electrophysiological signals, such as electrocardiographic imaging (see, e.g., FIG. 6).

FIG. 4 depicts an example of a 3D deformation generator 300 that can be implemented to provide an organ surface deformation model 302, which represents deformation of an organ over time. The deformation model 302 can be formed as a 3D mesh structure of an organ surface (or multiple organ surfaces) that varies spatially over time. For example, the organ is the heart or another anatomical structure. The 3D deformation generator 300 can generate the deformation model 302 as a real-time model (e.g., continually updated based on real-time EIT) to visualize real-time deformation of the organ over time. Alternatively, the 3D deformation generator 300 can generate the deformation model 302 for a time interval that repeats (e.g., a number of 3D image frames) periodically or is gated to a physiological function, such as the patient's respiration cycle or cardiac cycle.

In the example of FIG. 4, the 3D deformation generator 300 can be implemented (e.g., instructions executable by the processor 36) to generate the organ surface deformation model 302 based on 3D EIT image data 304 and 3D anatomical data 306. The 3D deformation generator 300 is an example of the deformation calculator 50. The deformation generator 300 can implement methods similar to the image generator 250, but based on time-varying 3D EIT image data that varies as a function of time, shown as 3D EIT image data(t) 304, which may also be referred to as four-dimensional image data. The 3D EIT image data(t) 304 can be acquired over one or more fixed time intervals or acquired continuously. For example, the 3D EIT image data(t) 304 can be generated as a series of image frames by the tomographic reconstruction method 46 based on electrical data 32 acquired over a prescribed time interval or continuously. The number and frame rate of the image frames in the 3D EIT image data(t) 304 can be fixed or variable responsive to a user input or monitored condition, such as described herein.

The 3D deformation generator 300 includes an edge extraction method 308 (e.g., instructions executable by the processor 36) to process the time-varying 3D EIT image data 304 and identify edges within each of the 3D image frames. The edges can represent boundaries between different tissue structures and/or bodily fluids (e.g., organ surfaces, vessel walls, or the like) in each image frame. For example, the edge extraction method 308 can be programmed to implement a variety of one or more edge detection methods, such as described herein. The edge extraction method 308 can also be programmed to perform smoothing of the edge detected data to provide smoothed version of the edge-detected image data at 310.

The image generator 250 also includes an image segmentation method 312 (e.g., instructions executable by the processor 36) to process 3D anatomical data 306 and provide segmented image data at 314 (which can be stored in memory for further processing). In some examples, the 3D anatomical data 256 can be 3D image (e.g., a high-resolution image acquired by a 3D imaging modality) for a region of interest of the patient's body, such as described herein. The image segmentation method 312 can implement various image segmentation methods as described herein.

The 3D deformation generator 300 includes a registration method 316 (e.g., instructions executable by the processor 36) programmed to fuse each frame of the edge-detected EIT image data 310 with the segmented image data 314 to provide 3D organ deformation data (also referred to as fused deformation images) 318 that varies as a function of time. As described with respect to the time-varying 3D EIT image data, the organ deformation data 318 varies over the same time (e.g., an interval or continuously over time). The registration method 316 can be programmed to preserve the organ surface boundaries for each image frame, which is enhanced by the 3D anatomical data 306 (e.g., at a higher spatial resolution). In some examples the registration between the image data 260 and 264 can utilize neural networks and/or other machine learning techniques.

In some examples, the 3D deformation generator 300 can also include a Laplace diffusion method 320. The Laplace diffusion method 320 applies the Laplace operator to deform the high resolution image (e.g., x-ray CT or MRI) with the deformation field, which is a displacement field (e.g., a displacement vector field) that constitutes part of or is derived from the EIT image data (e.g., one or more EIT heat maps). The Laplace diffusion method 320 provides an output based on the correspondence between the high resolution image and the displacement field, which can be represented by the organ surface deformation model 302. For example, the final result of the Laplace diffusion method is a real-time deformed version of the still medical image (e.g. MRI or CT) that wraps with the displacement field derived from the EIT. The resulting organ surface deformation model 302 can be rendered (e.g., by the processor 36) on an output device (e.g., display 56) for analysis by a health care provider. Also, or as an alternative, the organ surface deformation model 302 can be stored in memory for further processing. For example, the organ surface deformation model 302 can be used to generate a 3D time-varying anatomical model for surgical navigation (see, e.g., FIG. 5) and/or provide a 3D time-varying anatomical model (geometry data) for reconstruction of electrophysiological signals, such as electrocardiographic imaging (see, e.g., FIG. 6).

FIG. 5 depicts an example of a system 400 for combining 3D EIT geometry or image data 402 and navigation data 404, such for use in real-time surgical navigation of an object (e.g., a catheter or probe) within a region of interest that resides with the volume surrounded by body surface electrodes (e.g., electrodes 12). The system 400 thus can enable image data to be generated for visualizing in 3D space both anatomical structures through the object is traveling and position of the object relative to the anatomical structures.

In the example of FIG. 5, the system 400 includes a model generator 406 (e.g., instructions executable by the processor 36) to generate a 3D anatomical model 408 based on the 3D EIT geometry or image data 402. The 3D EIT geometry or image data 402 can be implemented as the 3D EIT image data 40 or the geometry/image data 252. Accordingly, the description of FIG. 5 can refer to certain aspects of FIGS. 1, 3, and 4. For example, the 3D anatomical model 408 can represent surfaces of one or more organs and/or vessel walls in 3D spatial coordinates, such as a coordinate system can be defined for or associated with the 3D volume within the region surrounded by an arrangement of body surface electrodes (e.g., electrodes 12) used to provide the 3D EIT geometry or image data 402. The 3D anatomical model 408 can represent a static geometry for one or more anatomical structures or it can be dynamic and vary over time (e.g., corresponding to the organ surface deformation model 302). Thus, in some examples, the 3D anatomical model 408 represents a real-time model representative of real time deformation of one or more anatomic structures, such as described herein.

The system 400 can include or be coupled to a navigation system 410 that is adapted to provide the navigation data 404. In some examples, the navigation system 410 is configured to localize a spatial position of an object (e.g., an invasive electrode 16) in a coordinate system of the navigation system 410. The navigation data 404 can be stored in memory and represent a real-time spatial position and orientation of the object within the coordinate system of the navigation system 410.

As an example, one or more invasive electrodes (e.g., electrodes 16) can be coupled to or otherwise carried by an electrophysiology probe. The probe can be a catheter that is moveable within the patient's body, such that the position of the probe and associated electrode(s) can vary. The probe may be moved manually, robotically assisted or fully robotically. For example, a cardiac catheter can be inserted into a femoral vein and advanced to a position within the patient's heart and/or along an outer surface of the patient's heart. The probe and electrode(s) can be configured to measure electrophysiological signals on a surface (endocardial or epicardial surface) of the patient's heart. Also, or as an alternative, the electrode(s) carried by the probe can be configured to emit or inject signals to the body, such as for stimulation (e.g., pacing or cardiac resynchronization therapy) or ablation.

The navigation data 404 thus represents a three-dimensional spatial position (e.g., spatial coordinates) of the electrode (e.g., invasive electrode 16). Alternatively, the navigation data 404 can represent the location of a sensor or other known location on the probe carrying the electrode(s), and the spatial location of each electrode and/or probe can be derived readily from the navigation data 404.

As described below, for example, a registration/mapping method 412 (e.g., instructions executable by the processor 36) can be programmed to register spatial location of the invasive electrode 16, which is described by or derived from the navigation data 404, with the 3D anatomical model 408. For example, the registration/mapping method 412 includes a transformation programmed to register the 3D anatomical model and the navigation data in a common coordinate system, which can be the coordinate system of the navigation system, the coordinate system of model (e.g., the patient's body), or another common coordinate system. The registration/mapping method 412 can repeat the registration (e.g., at a refresh rate). The refresh rate can be adjusted in response to detecting changes in the location data (e.g., based on the speed or velocity of the probe) as the electrode is moved within the patient's body. Alternatively, or additionally, the refresh rate can be adjusted in response to detecting deformation of anatomical structure and/or responsive to a user input instruction. In some examples, the navigation system 410 can also generate the navigation data 404 to include the location of one or more of the body surface electrodes 12, which are distributed across an outer surface of the patient's body (e.g., around the thorax).

Useful examples of the navigation system 410 include the STEALTHSTATION navigation system (commercially available from Medtronic Inc.), the CARTO XP EP navigation system (commercially available from Biosense-Webster) and the ENSITE NAVX visualization and navigation technology (commercially available from St. Jude Medical); although other tracking systems could be used to provide the navigation data 404 representative of the spatial position and orientation for the invasive electrode 16 and associated probe or catheter. Examples of other tracking systems include an electromagnetic tracking system, a magnetic tracking system, an optical tracking system, and the like.

The system 400 can include one or more image processing methods 414 (e.g., instructions executable by the processor 36) to process the registered model and navigation data and provide corresponding image data 416, which can be rendered on a display 418 (e.g., display 56). The system can generate the image data 416 at a refresh rate sufficient to provide a real-time 3D visualization that includes a graphical representation of the patient's anatomy, which can be static or dynamic, and a graphical representation of the object superimposed on the graphical representation of the patient's anatomy. The location of the object (e.g., one or more electrodes and/or catheter carrying the electrode(s)) in the visualization can show the current 3D spatial location and orientation of the object relative to the 3D representation of the patient's anatomy as the object moves within the patient's body. Thus, the system 400 can facilitate navigation of the object through the patient's anatomy (e.g., providing real-time feedback) to one or more target sites, such as to perform ablation or another treatment modality (e.g., pacing or resynchronization therapy).

FIG. 6 depicts an example of a system 450 for reconstructing electrophysiological signals on a surface of interest based on 3D EIT geometry or image data 452. The system 450 includes a model generator 454 (e.g., instructions executable by the processor 36) to generate geometry for one or more 3D surfaces of interest, shown at 456, based on the 3D EIT geometry or image data 452. The 3D EIT geometry or image data 452 can be implemented as the 3D EIT image data 40, the 3D geometry/image data 252, or the time varying 3D EIT image data 304. Accordingly, the description of FIG. 5 can refer to certain aspects of FIGS. 1, 3, and 4. The 3D surface of interest geometry data 456 can represent the geometry of the surface of interest onto which electrophysiological signals are to be reconstructed. For example, the 3D surface of interest geometry data 456 describes a 3D epicardial surface, a 3D endocardial surface, or both endocardial and epicardial surfaces. The 3D surface of interest geometry data 456 can represent a static geometry for one or more anatomical structures or it can represent a dynamic geometry that varies over time (e.g., corresponding to the organ surface deformation model 302). Thus, in some examples, the 3D anatomical model 408 represents a real-time model representative of real time deformation of one or more anatomic surfaces, such as described herein. In some examples, the 3D surface of interest geometry data 456 can be registered in the same spatial coordinate system as electrode geometry data 458, which represents a spatial position (e.g., 3D spatial coordinates) of an arrangement of electrodes that acquire electrophysiological signals from the patient's body. Appropriate anatomical or other landmarks, including locations for invasive electrodes (e.g., electrodes 12 and/or 16) can be identified in the electrode geometry data 458 to facilitate spatial registration of 3D surface of interest and the electrode geometry. The identification of such landmarks can be done manually (e.g., by a person via image editing software) or automatically (e.g., via image processing techniques).

The arrangement of electrodes can include non-invasive body surface electrodes (e.g., electrodes 12), one or more invasive electrodes (e.g., electrode(s) 16), or a combination of non-invasive body surface and invasive electrodes (e.g., 12 and 16). Thus, the electrode geometry data 458 (e.g., corresponding to the electrode geometry data 38 of FIG. 1) can represent the spatial position of body surface electrodes, invasive electrodes, or a combination thereof. The electrode geometry data 458 can be provided in a number of ways, such as described herein with respect to FIG. 1. The electrode geometry data 458 can be the provided for the electrodes using the same approach or different approaches can be used to provide the electrode geometry data for different subsets of the electrodes. For example, the electrode geometry data 458 for body surface electrodes can be determined in one way (e.g., extracted from 3D medical images, optical imaging, physical measurements, etc.) and the electrode geometry data 458 for invasive electrodes can be determined in a different way (e.g., by a navigation system). In the example when the electrode geometry data 458 representing the 3D spatial location for the body surface electrodes and the invasive electrodes are determined in different ways, the respective electrode geometry data can be registered in a common spatial coordinate system, such as by applying a corresponding spatial transformation.

The system 450 thus also receives (or otherwise accesses) EP measurement data 460, which is representative of respective some or all the EP signals measured by the electrodes. The EP measurement data 460 can represent electrical signals measured by the one or more (e.g., including up to all) of the electrodes (e.g., electrodes 12 and 16). The EP measurement data 460 can also include other associated data (e.g., metadata identifying each electrode that provides a signal measurement, timestamps describing the measurement or sample time, and the like. The spatial position for each of the electrodes (as provided by the electrode geometry data 458) can be linked to and/or stored with the EP measurement data 460 to provide corresponding electroanatomic data.

The system 450 also includes an EP reconstruction method 462 (e.g., instructions executable by the processor 36) programmed to compute reconstructed electrical signals for locations on a surface of interest within the patient's body. In one example, the processor executes the EP reconstruction method 462 to compute the reconstructed EP signals (e.g., electrical potentials) on the surface of interest (e.g., defined by the data 456) by executing instructions (an inverse algorithm) that solve the inverse problem based on geometry data 456 and 458 and the EP measurement data 460. Examples of inverse algorithms that can be implemented by the EP reconstruction method 462 include those disclosed in U.S. Pat. Nos. 6,772,004, 7,983,743 and 9,980,660. The EP reconstruction method 462 can implement other inverse algorithms in other examples. The EP reconstruction method 462 can calculate the reconstructed electrical signals on the surface of interest for one or more surfaces of interest over one or more time intervals. The time interval(s) may be selected through a user interface (e.g., user interface 52) in response to a user input entered by a user device (e.g., mouse, keyboard, touchscreen interface, gesture interface or the like).

As a further example, the EP reconstruction method 462 is programmed to calculate a transfer matrix based on the geometry data 456 and 458 and the EP measurement data 460. The EP reconstruction method 462 further may employ a regularization technique to estimate values for the reconstructed electrical signals on the surface of interest, which is defined by the 3D surface of interest geometry data 456.

In some examples, the surface of interest is a cardiac envelope, such as an epicardial surface, an endocardial surface, a combination of epicardial and endocardial surfaces of the patient's heart or other three-dimensional geometrical surface (e.g., a sphere). In other examples, the 3D surface of interest geometry data 456 represents the surfaces of interest as three-dimensional data describing a surface on to which reconstructed signals are computed (by the EP reconstruction method 462) and one or more surfaces where invasive measurements are made (e.g., by the invasive electrodes 16). Other surfaces of interest, such as part of the brain or other anatomical structures can be identified in other examples.

As a further example, the EP reconstruction method 462 is programmed to calibrate the heart-torso model to take into account inhomogeneity of the patient's body volume between the heart and the body surface electrode locations, in which the inhomogeneities correspond to impedance values represented in the 3D EIT geometry or image data 452 (e.g., EIT image data 40). In an example, model data can be calibrated based on the 3D EIT geometry or image data 452 to characterize inhomogeneity of a patient's body between the heart and the body surface electrode locations. As a further example, inhomogeneities may be selectively applied to the part of a heart-torso model that represents conductivity of the patient's body, such that some of the volume-body conductor may be represented as a homogeneous conductor and other portions of the volume-body conductor represented by the model may reflect inhomogeneities. The EP reconstruction method 462 thus may compute reconstructed electrical signals on the surface of interest using the calibrated torso model data.

The system 450 can also include an output generator 464 that is programmed to generate an image data 466 (e.g., representing an EP map) that can be rendered on a display 468 to graphically visualize the reconstructed electrical signal on the surface of interest. As disclosed herein, the surface of interest may be an epicardial surface, an endocardial surface, or a combination of epicardial or endocardial surfaces. Additionally, the surface of interest can be a cardiac envelope, such as a surface residing between the center of a patient's heart and the body surface where the electrodes are positioned. The surface of interest may encompass the entire cardiac surface or one or more surface regions (epicardial or endocardial) such as described herein.

The output generator 464 thus provides image data 466 that may be provided to the display 468 to visualize one or more 3D electrocardiographic maps as well as other electrical information derived from the reconstructed electrical signals. For example, the output generator 464 is programmed to generate image data 466, such as to provide an EP map based on the reconstructed signals (generated by EP reconstruction method 462) and invasively measured electrical information (e.g., from invasive electrode(s) 16). By including reconstructed electrical signals and actual signals (measured invasively) in a combined EP map, the combined EP map concurrently provides respective global and local assessments in the EP map. Additionally, or alternatively, the reconstructed electrical signal on surface of interest may further be enhanced through electrical signals acquired concurrently by invasive measurements.

By way of further example, the output generator 464 can provide the image data 466 based on the reconstructed EP signals (provided by the EP reconstruction method 462) and other data 470. The other data 470 can include navigation data (e.g., navigation data 404), data defining one or more viewing angles for visualization, data selecting one or more times preceding time intervals, and/or instructions to provide a live real-time visualization, etc. Depending on the type and content of the other data 470, the output generator 464 can thus update the image data 466 accordingly.

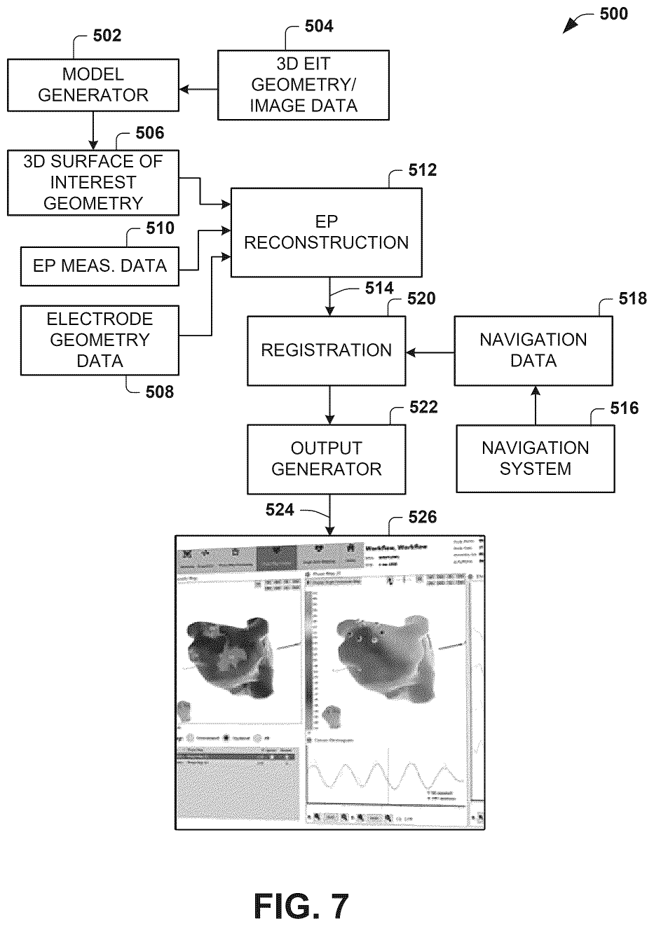

FIG. 7 depicts an example of a system 500 that combines surgical navigation and reconstructed electrophysiological signals. For example, the system 500 can be considered a combination of the system 400 of FIG. 5 and the system 450 of FIG. 6 further in view of the system of FIG. 1. Accordingly, the description of FIG. 7 may also refer to certain aspects of FIGS. 1, 5, and 6.

The system 500 includes a model generator 502 (e.g., instructions executable by the processor 36) to generate 3D surface of interest geometry data 506 for one or more 3D surfaces of interest based on the 3D EIT geometry or image data 504. The 3D EIT geometry or image data 504 can be implemented as or derived from the 3D EIT image data 40, the 3D geometry/image data 252, or the time varying 3D EIT image data 304, as described herein. The 3D surface of interest geometry data 506 can represent the geometry of the surface (or surfaces) of interest onto which electrophysiological signals are to be reconstructed. As described herein, the 3D surface of interest geometry 506 can be static (e.g., fixed spatially) or dynamic (e.g., time-varying to represent 3D deformation of the anatomic surfaces over time). The 3D surface of interest geometry data 506 can be spatially registered in the same spatial coordinate system as electrode geometry data 508. The electrode geometry data 508 represents a spatial position (e.g., 3D spatial coordinates) of an arrangement of electrodes, which can include non-invasive body surface electrodes (e.g., electrodes 12), one or more invasive electrodes (e.g., electrode(s) 16), or a combination of non-invasive body surface and invasive electrodes (e.g., 12 and 16). Thus, the electrode geometry data 508 (e.g., corresponding to the electrode geometry data 38 of FIG. 1) can represent the spatial position of body surface electrodes, invasive electrodes, or a combination thereof. The electrode geometry data 508 can be determined differently for different subsets of the electrodes (e.g., in examples where one or more electrodes are invasive, and the other electrodes are body surface electrodes) or a common method can be used for all electrodes.

As a further example, the electrodes (e.g., electrodes having locations represented by the electrode geometry data 508) are configured to measure electrophysiological signals from the patient's body and provide EP measurement data 510. The EP measurement data 510 thus can represent electrical signals measured by the one or more (e.g., including up to all) of the electrodes (e.g., electrodes 12 and 16). The EP measurement data 510 can also include other associated data (e.g., metadata identifying each electrode that provides a signal measurement, timestamps describing the measurement or sample time, and the like. The spatial position for each of the electrodes (as provided by the electrode geometry data 508), which can be fixed or change over time, can be linked to and/or stored with the EP measurement data 510 to provide corresponding electroanatomic data.

The system 500 also includes an EP reconstruction method 512 (e.g., instructions executable by the processor 36) programmed to compute reconstructed EP signals 514 for locations on the surface of interest (e.g., defined by the 3D surface of interest geometry data 506) based on the 3D surface of interest geometry data 506, the electrode geometry data 508, and the EP measurement data 510. As described herein, for example, the processor executes the EP reconstruction method 512 to compute the reconstructed EP signals (e.g., electrical potentials) 514 on the surface of interest by executing instructions (an inverse algorithm) that solve the inverse problem based on geometry data 506 and 508 and the EP measurement data 510.

In the example of FIG. 7, the system 500 also includes a navigation system 516 that is adapted to provide the navigation data 518. In some examples, the navigation system 410 is configured to localize a 3D spatial position of an object (e.g., an invasive electrode 16) in a coordinate system of the navigation system 516. The navigation data 518 thus represents a three-dimensional spatial position (e.g., spatial coordinates) of the electrode (e.g., invasive electrode 16). Alternatively, the navigation data 518 can represent the location of a sensor or other known location on the probe carrying the electrode(s), and the spatial location of each electrode and/or probe can be derived readily from the navigation data 518. In some examples, the navigation system 516 can also generate the navigation data 518 to include the location of one or more of the body surface electrodes 12, which are distributed across an outer surface of the patient's body (e.g., on the thorax), which can thus provide the electrode geometry data 508. The navigation data 518 can be stored in memory and represent a real-time spatial position and orientation of the object within the coordinate system of the navigation system 516.

The system 500 also includes a registration method 520 (e.g., instructions executable by the processor 36) programmed to register spatial location of the invasive electrode 16, which is described by or derived from the navigation data 518, with the 3D surface(s) of interest onto which the EP signals have been reconstructed. For example, the registration method 520 includes a transformation programmed to register the 3D surface geometry data and the navigation data 518 in a common coordinate system. The common coordinate system can be the coordinate system of the navigation system, the coordinate system of surface of interest (e.g., the patient's body), or another common coordinate system. While the registration method 520 is shown to be applied to the reconstructed EP signals 514, in other examples, the registration method 520 can be implemented to register the navigation data 518 with the 3D surface of interest geometry data 506 prior to or as part of the EP reconstruction method 512.

The registration method 520 can repeat the registration (e.g., at a refresh rate) so the position of electrode(s) being localized are being tracked in real-time or near real-time. In one example, the refresh rate can be adjusted in response to detecting changes in the navigation data 518 (e.g., based on the speed or velocity of the probe) as the electrode is moved within the patient's body. Alternatively, or additionally, the refresh rate can be adjusted in response to detecting deformation of anatomical structure. Also, or as an alternative, the refresh rate can be adjusted responsive to a user input instruction.

The system 500 can also include an output generator 522 that is programmed to generate output data 524 based on reconstructed EP signals 514 and the navigation data 518, which are spatially registered (e.g., by registration method 520). The output data 524 can be provided to a display 526 to graphically visualize the reconstructed EP signals and one or more electrodes (or probe carrying the electrode(s)) relative to the 3D surface of interest. For example, the output generator 522 can provide the output data 524 for visualizing in 3D space (i) one or more anatomical structures (e.g., 3D surfaces of interest) through which an electrode/probe is traveling, (ii) the position and orientation of the electrode/probe relative to the anatomical structures, and (iii) EP signals reconstructed onto the anatomical structures. The visualization thus can provide the user with real-time feedback on the spatial position of the one or more electrodes (or probe carrying the electrode(s)) and the effects of interaction between the electrode(s) or other parts of the probe and the patient's anatomy. The effects of such interaction can include deformation of the surface of interest responsive to contact between the probe and the surface of interest, in which such deformation is represented in the 3D EIT geometry or image data 504. The effects can also result in EP signals across the surface of interest, which are represented in the EP measurement data 510 and reconstructed onto the surface of interest.

As disclosed herein, the surface of interest may be an epicardial surface, an endocardial surface, or a combination of epicardial or endocardial surfaces. Additionally, the surface of interest can be a cardiac envelope, such as a virtual surface residing between the center of a patient's heart and the body surface where the electrodes are positioned. Also, or as alternative, the surface of interest may encompass the entire cardiac surface or one or more surface regions (epicardial or endocardial) such as described herein.

As a further example, one or more invasive electrodes (e.g., electrodes 16) can be coupled to or otherwise carried by a probe. The probe can be a catheter that is moveable within the patient's body, such that the position of the probe and associated electrode(s) can vary relative to the patient's body. The probe may be moved manually, robotically assisted or fully robotically. The probe and electrode(s) can be configured to measure electrophysiological signals on a surface (endocardial or epicardial surface) of the patient's heart. Also, or as an alternative, the electrode or other devices carried by the probe can perform pacing (e.g., cardiac resynchronization therapy (CRT)) or other forms of cardiac rhythm therapy (e.g., cardioversion and/or defibrillation). In another example, the one or more electrode or other devices carried by the probe can be adapted to perform ablation, which can include irreversible electroporation (IRE) of tissue, reversible electroporation (RE), as well as other types of ablation (e.g., cryoablation, RF ablation). In examples, where such treatment is to be implemented, the output generator 522 can update the output data 524 to provide real-time feedback representing the reconstructed EP data on the surface of interest (based on the EP measurement data 510) and changes thereof responsive to the treatment being applied at a particular location, which is also visualized concurrently as part of the output (based on the navigation data 518) that is shown on the display 526. The output data 524 provided to the display 526 further can include and/or be derived from the 3D EIT geometry or image data 504. Accordingly, real-time physiological changes including deformation thereof in the surface interest (e.g., heart or other organ surfaces) can also be visualized along with a graphical representation of a surgical tool that is being tracked by the navigation system 516. The systems and methods herein can facilitate surgical navigation in a variety of applications. Some examples of procedures that can be enabled by 3D guidance provided by the system 500 include cardiac ablation, valve implantation, cardiac lead implantation, endoluminal lung surgery, upper gastrointestinal surgery, left atrial appendage occlusion device implantation, directional atheroctomy procedure, renal denervation, and hysteroscopy. The systems and methods can be used to enable other types of procedures.

FIG. 8 depicts one example of a sensor apparatus 600 that may be attached around an individual's thorax for noninvasively sensing body surface electrical signals. The example sensor apparatus 600 may be configured according to the embodiments disclosed in U.S. Pat. No. 9,655,561, which is incorporated herein by reference. Other forms and arrangements of electrodes may be used in other examples, such as including the sensor apparatus disclosed in EP Patent No. 2352421.

The sensor apparatus 600 is dimensioned and configured to be applied to a torso of a patient (e.g., a human patient); however, different configurations can be utilized depending on the patient (e.g., could be human or other animal) and the particular type of electrophysiology to be performed. The sensor apparatus 600 can come in a plurality of sizes to accommodate a range of patient's sizes and body types.

The sensor apparatus 600 may include one or more substrate layers 612 that are formed of a flexible material adapted to conform over the surface of the patient's body. The substrate layer 612 provides an electrode-carrying substrate layer. In the example of FIG. 8, the sensor apparatus 600 includes an arrangement of electrodes 616 disposed on a contact surface of a corresponding electrode receiving portion of the substrate layer 612. Respective electrodes 616 can operate as sensors for measuring electrical activity. Additionally, or alternatively, electrodes 616 can be configured to deliver electrical energy (e.g., electrical current), as described herein. The electrodes 616 are coupled to a respective connector 620 through electrically conductive element (e.g., a trace or wire). The respective connectors 620 are adapted to couple to an electrode interface (e.g., electrode interface circuit 18), which may be a direct connection or a connection through additional cabling, to carry measured electrical signals or applied electrical signals between the electrode interface circuit and respective electrodes 616.

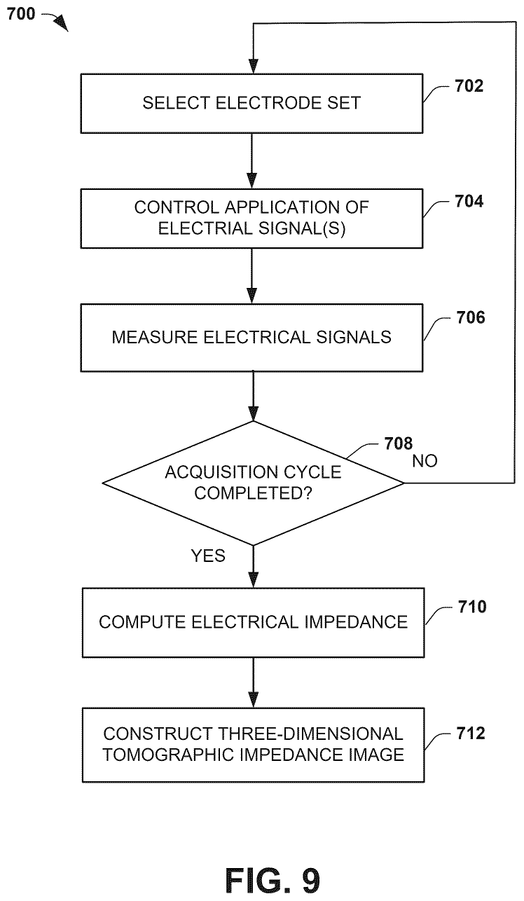

In view of the foregoing structural and functional features described above, example methods that can be implemented will be better appreciated with reference to the flow diagram of FIG. 9. While, for purposes of simplicity of explanation, the method 700 of FIG. 9 is shown and described as executing serially, it is to be understood and appreciated that such methods are not limited by the illustrated order, as some aspects could, in other examples, occur in different orders and/or concurrently with other aspects from that disclosed herein. Moreover, not all illustrated features may be required to implement a method. The methods or portions thereof can be implemented as instructions stored in one or more non-transitory machine readable media and be executed by a processor of one or more computer devices, for example. The method 700 can be implemented by the systems described herein, including FIGS. 1-7. Accordingly, the method 700 may refer to certain aspects of FIGS. 1-7. At 702, the method 700 includes selecting an electrode set that includes at least one source electrode and multiple measurement electrodes from a plurality of electrodes (e.g., electrodes 12 and/or 16) distributed across an individual's body. At 704, the method includes controlling application of an electrical signal to at least one source electrode of the selected electrode set for a respective phase of an acquisition cycle that includes a plurality phases. At 706, electrical signals from the measurement electrodes of the selected electrode set are measured responsive to the applied electrical signal for the respective phase of the acquisition cycle.

At 708, a determination is made as to whether the acquisition cycle has completed. If the determination is negative (NO), the metho returns to 702 for repeating the selecting, the controlling and the measuring for each other phase of the acquisition cycle to provide electrical data representative of the applied electrical signals and the measured electrical signals for the acquisition cycle. If the determination at 708 is positive (YES), the method proceeds to 710.

At 710, the method includes computing (e.g., by processor 36) an electrical impedance between the at least one source electrode and each of the measurement electrodes based on the electrical data for each phase of the acquisition cycle. At 712, the method includes constructing a three-dimensional tomographic impedance image of the individual's body for the acquisition cycle based on the computed electrical impedances and geometry data. The geometry data represents a three-dimensional position of each of the plurality of electrodes (e.g., source and measurement electrodes 12 and 16) distributed across the individual's body.

In some examples, the method 700 can also include combining the three-dimensional tomographic impedance image with medical imaging data to define a fused three-dimensional image of patient anatomy (see, e.g., FIGS. 3 and 3). The medical imaging data can represent a three-dimensional medical image (e.g., CT, MRI, ultrasound) of a portion of the patient's body acquired by a three-dimensional medical imaging modality.

Also, or as an alternative, the method 700 can include segmenting the three-dimensional tomographic impedance image to define anatomical data representative of geometry for a 3D surface of interest within the patient's body. The geometry can further be used to provide a model of the geometry for the 3D surface of interest, such as described herein (see, e.g., FIGS. 3, 4, and/or 5). In some examples, the method 700 can further include reconstructing electrophysiological signals at locations across the surface of interest based on electrophysiological signals measured by the plurality of electrodes and the anatomical data.

Also, or as an alternative, the method 700 can also include receiving navigation data representative of a three-dimensional position of an object within the patient's body in a spatial domain of a navigation system (see, e.g., FIGS. 5 and/or 7). The three-dimensional position of the object can be spatially registered in a common spatial domain with the three-dimensional surface of interest. Output data can be generated for displaying a graphical representation of the object superimposed on the three-dimensional surface of interest based on the spatial registration.

### EXAMPLE EMBODIMENTS

Several aspects of the present technology are set forth in the following numbered examples.

### Example 1

A system comprising:
a plurality of electrodes adapted to be distributed in a three-dimensional arrangement relative to a patient's body;
an electrode interface circuit coupled to each of the plurality of electrodes and configured to one of apply an electrical signal to or measure an electrical signal from each the plurality of electrodes;
a controller configured to control the electrode interface circuit during an acquisition cycle to select which of the plurality of electrodes are source electrodes and which of the plurality of electrodes are measurement electrodes during respective phases of the acquisition cycle, in which each source electrode is configured to apply a source electrical signal to the patient's body and each of the measurement electrodes is configured to measure an electrical signal responsive to the applied electrical signal during a respective phase of the acquisition cycle;
   non-transitory memory to store instructions and data, in which the data comprises electrical data representative of the measured electrical signals and the applied electrical signals for the acquisition cycle, and geometry data representative of a three-dimensional position of each of the plurality of electrodes relative to the patient's body; and
   a processor coupled to the memory to access the data and instructions stored in the memory, the instructions, when executed by the processor, cause the processor to at least:
      compute an electrical impedance between the source electrode and each of the measurement electrodes based on the electrical data for each of the respective phases of the acquisition cycle; and
      construct a three-dimensional tomographic impedance image of the patient's body for the acquisition cycle based on the computed electrical impedances and the geometry data.

### Example 2

The system of example 1, wherein at least one source electrode and the measurement electrodes, as selected by the controller for each respective phase of the acquisition cycle, define an electrode set for the respective phase, and at least some of the plurality of electrodes in each electrode set are non-coplanar.

### Example 3

The system of example 1 or 2, wherein the applied electrical signal is a current signal having a frequency and therefore a power spectrum in the complex domain.

### Example 4

The system of example 3, wherein the controller is configured to control the electrode interface circuit to apply current signals having different frequencies to respective source electrodes during the respective phases of the acquisition cycle.

### Example 5

The system of example 4, wherein the current signals are applied to each of the source electrodes sequentially or concurrently at the different frequencies during the respective phases of the acquisition cycle.

### Example 6

The system according to any one of the preceding examples, wherein each acquisition cycle includes a number of the respective phases that depends on a number of the plurality of electrodes and a number of source electrodes selected for each respective phase.

### Example 7

The system according to any one of the preceding examples, wherein the plurality of electrodes comprises:
a noninvasive arrangement of electrodes adapted to be placed on an outer surface of the patient's body; and
an invasive electrode carried by a probe and adapted to be positioned within the patient's body, in which the invasive electrode defines a respective source electrode during at least one phase of the acquisition cycle.

### Example 8

The system of example 7, wherein the instructions are further programmed to cause the processor to localize the probe and/or the invasive electrode within the patient's body based on the three-dimensional tomographic impedance image of the patient's body.

### Example 9

The system according to any one of the preceding examples, wherein the instructions are further programmed to cause the processor to:
segment the three-dimensional tomographic impedance image to define anatomical data representative of geometry for a surface of interest within the patient's body; and
reconstruct electrophysiological signals at locations across the surface of interest based on electrophysiological signals measured by the plurality of electrodes and the anatomical data.

### Example 10

The system according to any one of the preceding examples, wherein the instructions are further programmed to cause the processor to combine the three-dimensional tomographic impedance image with medical imaging data to define a fused three-dimensional image of patient anatomy, in which the medical imaging data is representative of a three-dimensional medical image of a portion of the patient's body acquired by a three-dimensional medical imaging modality.

### Example 11

The system of example 10, wherein the instructions are further programmed to cause the processor to repeatedly construct the three-dimensional tomographic impedance image based on electrical data acquired over each of a plurality of acquisition cycles and the three-dimensional tomographic impedance image varies over time to represent movement and/or deformation of structures within the patient's body over at least one time interval.

### Example 12

The system of example 11, wherein the instructions are further programmed to cause the processor to control a refresh rate at which the three-dimensional tomographic impedance image is constructed based on at least one of the movement and/or deformation of the structures within the patient's body and a location of an object within the patient's body, in which the location of the object within the patient's body is determined based on analyzing of the three-dimensional tomographic impedance image.

### Example 13

The system of example 10, wherein the instructions are further programmed to cause the processor to infer anatomical geometry within the patient's body based on the three-dimensional tomographic impedance image and the imaging data.

### Example 14

The system according to any preceding example, wherein the instructions are further programmed to cause the processor to calculate resolution of the three-dimensional tomographic impedance image.

### Example 15

The system according to any preceding example, wherein the electrical impedance is computed as a value having a real component and an imaginary component.

### Example 16

A computer-implemented method comprising:
selecting an electrode set that includes at least one source electrode and multiple measurement electrodes from a plurality of electrodes distributed across an individual's body;
controlling application of an electrical signal to the at least one source electrode of the selected electrode set for a respective phase of an acquisition cycle that includes a plurality phases;
measuring electrical signals from the measurement electrodes of the selected electrode set responsive to the applied electrical signal for the respective phase of the acquisition cycle;
repeating the selecting, the controlling and the measuring for each other phase of the acquisition cycle to provide electrical data representative of the applied electrical signals and the measured electrical signals for the acquisition cycle;
computing, by a processor, an electrical impedance between the at least one source electrode and each of the measurement electrodes based on the electrical data for each phase of the acquisition cycle; and
constructing, by the processor, a three-dimensional tomographic impedance image of the individual's body for the acquisition cycle based on the computed electrical impedances and geometry data, in which the geometry data represents a three-dimensional position of each of the plurality of electrodes distributed across the individual's body.

### Example 17

The method of example 16, further comprising:
combining the three-dimensional tomographic impedance image with medical imaging data to define a fused three-dimensional image of patient anatomy, in which the medical imaging data is representative of a three-dimensional medical image of a portion of the individual's body acquired by a three-dimensional medical imaging modality.

### Example 18

The method of example 16 or 17, further comprising:
segmenting the three-dimensional tomographic impedance image to define anatomical data representative of geometry for a three-dimensional surface of interest within the individual's body; and
reconstructing electrophysiological signals at locations across the surface of interest based on electrophysiological signals measured by the plurality of electrodes and the anatomical data.

### Example 19

The method of example 18, further comprising:
receiving navigation data representative of a three-dimensional position of an object within the individual's body in a spatial domain of a navigation system;
spatially registering the three-dimensional position of the object in a common spatial domain with the three-dimensional surface of interest; and
generating output data for displaying a graphical representation of the object superimposed on the three-dimensional surface of interest based on the spatial registration.

### Example 20

A non-transitory to store data and instructions that, when executed by a processor, cause the processor to perform a method comprising:
selecting an electrode set that includes at least one source electrode and multiple measurement electrodes from a plurality of electrodes distributed across an individual's body;
controlling application of an electrical signal to the at least one source electrode of the selected electrode set for a respective phase of an acquisition cycle that includes a plurality phases;
receiving measured electrical signal from the measurement electrodes of the selected electrode set responsive to the applied electrical signal for the respective phase of the acquisition cycle;
repeating the selecting, the controlling and the storing for each other phase of the acquisition cycle to store electrical data representative of the applied electrical signals and the measured electrical signals for the acquisition cycle;
computing an electrical impedance between the at least one source electrode and each of the measurement electrodes based on the electrical data for each phase of the acquisition cycle; and
constructing a three-dimensional tomographic impedance image of the individual's body for the acquisition cycle based on the computed electrical impedances and geometry data, in which the geometry data represents a three-dimensional position of each of the plurality of electrodes distributed across the individual's body.

In view of the foregoing structural and functional description, those skilled in the art will appreciate that portions of the invention may be embodied as a method, data processing system, or computer program product. Accordingly, these portions of the present invention may take the form of an entirely hardware embodiment, an entirely software embodiment, or an embodiment combining software and hardware. Furthermore, portions of the invention may be a computer program product on a computer-usable storage medium having computer readable program code on the medium. Any suitable computer-readable medium may be utilized including, but not limited to, static and dynamic storage devices, hard disks, optical storage devices, and magnetic storage devices.

Certain embodiments of the invention have also been described herein with reference to block illustrations of methods, systems, and computer program products. It will be understood that blocks of the illustrations, and combinations of blocks in the illustrations, can be implemented by computer-executable instructions. These computer-executable instructions may be provided to one or more processor of a general purpose computer, special purpose computer, or other programmable data processing apparatus (or a combination of devices and circuits) to produce a machine, such that the instructions, which execute via the processor, implement the functions specified in the block or blocks.

These computer-executable instructions may also be stored in computer-readable memory that can direct a computer or other programmable data processing apparatus to function in a particular manner, such that the instructions stored in the computer-readable memory result in an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

What have been described above are examples. It is, of course, not possible to describe every conceivable combination of components or methods, but one of ordinary skill in the art will recognize that many further combinations and permutations are possible. Accordingly, the invention is intended to embrace all such alterations, modifications, and variations that fall within the scope of this application, including the appended claims. Where the disclosure or claims recite “a,” “an,” “a first,” or “another” element, or the equivalent thereof, it should be interpreted to include one or more than one such element, neither requiring nor excluding two or more such elements. As used herein, the term “includes” means includes but not limited to, the term “including” means including but not limited to. The term “based on” means based at least in part on.

What is claimed is:

1. A system comprising:
a plurality of electrodes adapted to be distributed in a three-dimensional arrangement relative to a patient's body;
an electrode interface circuit coupled to each of the plurality of electrodes and configured to one of apply an electrical signal to or measure an electrical signal from each the plurality of electrodes;
a controller configured to control the electrode interface circuit during an acquisition cycle to select which of the plurality of electrodes are source electrodes and which of the plurality of electrodes are measurement electrodes during respective phases of the acquisition cycle, in which each source electrode is configured to apply a source electrical signal to the patient's body and each of the measurement electrodes is configured to measure an electrical signal responsive to the applied electrical signal during a respective phase of the acquisition cycle;
non-transitory memory to store instructions and data, in which the data comprises electrical data representative of the measured electrical signals and the applied electrical signals for the acquisition cycle, and geometry data representative of a three-dimensional position of each of the plurality of electrodes relative to the patient's body; and
a processor coupled to the memory to access the data and instructions stored in the memory, the instructions, when executed by the processor, cause the processor to at least:
compute an electrical impedance between the source electrode and each of the measurement electrodes based on the electrical data for each of the respective phases of the acquisition cycle; and
construct a three-dimensional tomographic impedance image of the patient's body for the acquisition cycle based on the computed electrical impedances and the geometry data.

2. The system of claim 1, wherein at least one source electrode and the measurement electrodes, as selected by the controller for each respective phase of the acquisition cycle, define an electrode set for the respective phase, and at least some of the plurality of electrodes in each electrode set are non-coplanar.

3. The system of claim 1, wherein the applied electrical signal is a current signal having a frequency and a power spectrum in the complex domain.

4. The system of claim 3, wherein the controller is configured to control the electrode interface circuit to apply current signals having different frequencies to respective source electrodes during the respective phases of the acquisition cycle.

5. The system of claim 4, wherein the current signals are applied to each of the source electrodes sequentially or concurrently at the different frequencies during the respective phases of the acquisition cycle.

6. The system of claim 1, wherein each acquisition cycle includes a number of the respective phases that depends on a number of the plurality of electrodes and a number of source electrodes selected for each respective phase.

7. The system of claim 1, wherein the plurality of electrodes comprises:
a noninvasive arrangement of electrodes adapted to be placed on an outer surface of the patient's body; and
an invasive electrode carried by a probe and adapted to be positioned within the patient's body, in which the invasive electrode defines a respective source electrode during at least one phase of the acquisition cycle.

8. The system of claim 7, wherein the instructions are further programmed to cause the processor to localize the probe and/or the invasive electrode within the patient's body based on the three-dimensional tomographic impedance image of the patient's body.

9. The system of claim 1, wherein the instructions are further programmed to cause the processor to:
segment the three-dimensional tomographic impedance image to define anatomical data representative of geometry for a surface of interest within the patient's body; and
reconstruct electrophysiological signals at locations across the surface of interest based on electrophysiological signals measured by the plurality of electrodes and the anatomical data.

10. The system of claim 1, wherein the instructions are further programmed to cause the processor to combine the three-dimensional tomographic impedance image with medical imaging data to define a fused three-dimensional image of patient anatomy, in which the medical imaging data is representative of a three-dimensional medical image of a portion of the patient's body acquired by a three-dimensional medical imaging modality.

11. The system of claim 10, wherein the instructions are further programmed to cause the processor to repeatedly construct the three-dimensional tomographic impedance image based on electrical data acquired over each of a plurality of acquisition cycles and the three-dimensional tomographic impedance image varies over time to represent movement and/or deformation of structures within the patient's body over at least one time interval.

12. The system of claim 11, wherein the instructions are further programmed to cause the processor to control a refresh rate at which the three-dimensional tomographic impedance image is constructed based on at least one of the movement and/or deformation of the structures within the patient's body and a location of an object within the patient's body, in which the location of the object within the patient's body is determined based on analyzing of the three-dimensional tomographic impedance image.

13. The system of claim 10, wherein the instructions are further programmed to cause the processor to infer anatomical geometry within the patient's body based on the three-dimensional tomographic impedance image and the imaging data.

14. The system of claim 1, wherein the instructions are further programmed to cause the processor to calculate resolution of the three-dimensional tomographic impedance image.

15. The system of claim 1, wherein the electrical impedance is computed as a value having a real component and an imaginary component.

16. A computer-implemented method comprising:
selecting an electrode set that includes at least one source electrode and multiple measurement electrodes from a plurality of electrodes distributed across an individual's body;
controlling application of an electrical signal to the at least one source electrode of the selected electrode set for a respective phase of an acquisition cycle that includes a plurality phases;
measuring electrical signals from the measurement electrodes of the selected electrode set responsive to the applied electrical signal for the respective phase of the acquisition cycle;
repeating the selecting, the controlling and the measuring for each other phase of the acquisition cycle to provide electrical data representative of the applied electrical signals and the measured electrical signals for the acquisition cycle;
computing, by a processor, an electrical impedance between the at least one source electrode and each of the measurement electrodes based on the electrical data for each phase of the acquisition cycle; and
constructing, by the processor, a three-dimensional tomographic impedance image of the individual's body for the acquisition cycle based on the computed electrical impedances and geometry data, in which the geometry data represents a three-dimensional position of each of the plurality of electrodes distributed across the individual's body.

17. The method of claim 16, further comprising:
combining the three-dimensional tomographic impedance image with medical imaging data to define a fused three-dimensional image of patient anatomy, in which the medical imaging data is representative of a three-dimensional medical image of a portion of the individual's body acquired by a three-dimensional medical imaging modality.

18. The method of claim 16, further comprising:
segmenting the three-dimensional tomographic impedance image to define anatomical data representative of geometry for a three-dimensional surface of interest within the individual's body; and
reconstructing electrophysiological signals at locations across the surface of interest based on electrophysiological signals measured by the plurality of electrodes and the anatomical data.

19. The method of claim 18, further comprising:
receiving navigation data representative of a three-dimensional position of an object within the individual's body in a spatial domain of a navigation system;
spatially registering the three-dimensional position of the object in a common spatial domain with the three-dimensional surface of interest; and
generating output data for displaying a graphical representation of the object superimposed on the three-dimensional surface of interest based on the spatial registration.

20. A non-transitory to store data and instructions that, when executed by a processor, cause the processor to perform a method comprising:
selecting an electrode set that includes at least one source electrode and multiple measurement electrodes from a plurality of electrodes distributed across an individual's body;
controlling application of an electrical signal to the at least one source electrode of the selected electrode set for a respective phase of an acquisition cycle that includes a plurality phases;
receiving measured electrical signal from the measurement electrodes of the selected electrode set responsive to the applied electrical signal for the respective phase of the acquisition cycle;
repeating the selecting, the controlling and the storing for each other phase of the acquisition cycle to store electrical data representative of the applied electrical signals and the measured electrical signals for the acquisition cycle;
computing an electrical impedance between the at least one source electrode and each of the measurement electrodes based on the electrical data for each phase of the acquisition cycle; and
constructing a three-dimensional tomographic impedance image of the individual's body for the acquisition cycle based on the computed electrical impedances and geometry data, in which the geometry data represents a three-dimensional position of each of the plurality of electrodes distributed across the individual's body.
